(19) United States
(12) Patent Application Publication
(10) Pub. No.: US 2017/0058364 A1
(43) Pub. Date: Mar. 2, 2017

(54) METHODS AND KITS FOR SEQUENCING AND CHARACTERIZING PROTOZOA

(71) Applicant: Fry Laboratories, LLC, Scottsdale, AZ (US)

(72) Inventors: Stephen E. Fry, Scottsdale, AZ (US); Jeremy Ellis, Mesa, AZ (US)

(21) Appl. No.: 14/834,397

(22) Filed: Aug. 24, 2015

(51) Int. Cl. C12Q 1/68; G06F 19/24; G06F 19/22

(52) U.S. Cl. CPC C12Q 1/6893; G06F 19/22; G06F 19/24

(57) ABSTRACT

Disclosed are compositions, kits, and methods for detecting, characterizing, and/or identifying one or more protozoa. Various types of polymerase chain reaction techniques in connection with specifically designed primers can be used to detect a variety of, e.g., pathogenic, protozoa in samples.

### INCORPORATION BY REFERENCE OF SEQUENCE LISTING

Incorporated by reference in its entirety herein is a computer-readable nucleotide/amino acid sequence listing submitted concurrently herewith and identified as follows: One 12,288 byte ASCII (text) file named “6558801000_SequenceListing” created on Aug. 24, 2015.

### FIELD OF DISCLOSURE

The present disclosure generally relates to protozoa detection and characterization techniques. More particularly, various embodiments of the disclosure relate to methods, compositions, assays, and kits for the extraction, detection, identification, characterization, and/or reporting of protozoa.

### BACKGROUND OF THE DISCLOSURE

Various protozoa can contribute to diseases in animals, including humans. Unfortunately, suitable diagnostic methods and kits do not exist that allow for rapid detection and characterization of protozoa that may be present within a sample, such as a biological or other sample. As a result, methods of preventing, diagnosing, and/or treating such diseases may not be available or may be delayed, resulting in poor patient outcomes.

Accordingly, improved methods and kits for rapidly detecting, identifying, and/or characterizing protozoa are desired.

The above discussion of problems and solutions and any other discussions disclosed in this disclosure in relation to the related art is included solely for the purposes of providing a context for the present invention and should not be taken as an admission that any or all of the discussion was known at the time the invention was made.

### SUMMARY OF THE DISCLOSURE

Exemplary embodiments of the present disclosure are directed towards methods of and kits for detecting and/or characterizing protozoa. While the ways in which the disclosure addresses various shortcomings of the prior art are discussed in more detail below, in general, the methods and kits described herein can be used to rapidly and accurately characterize protozoa—e.g., either by identifying the species, by determining a species that has a relatively similar nucleic acid sequence to the protozoa—or characterizing other taxonomical classification(s) of the protozoa.

In accordance with various embodiments of the disclosure, a method of characterizing one or more protozoa includes the steps of generating a plurality of nucleic acid segments from a sample using one or more degenerate primers to form a pool of nucleic acid segments having a target region, sequencing the pool of nucleic acid segments to form sequences, and using a computer, characterizing the one or more protozoa. In accordance with exemplary aspects of these embodiments, the step of characterizing includes identifying the protozoa or identifying the nearest previously identified or known protozoa in a library. The identifying can be based on taxonomic categories, such as species, genus, or higher order classification of protozoa. In accordance with further aspects, the step of generating includes use of polymerase chain reaction (PCR), which can generate nucleic acid segments with one or more conserved regions and/or semi-conserved regions. By way of examples, forward and/or reverse primers with degenerate bases can be used with PCR to create a pool of nucleic acid segments that can be sequenced and analyzed, using a computer, to characterize the protozoa. In accordance with further aspects of these embodiments, the step of generating includes amplifying a nucleic acid segment corresponding to a first region of an 18S rRNA gene. Additionally or alternatively, one or more of the primer bases can be artificial or non-canonical. Exemplary methods can additionally or alternatively amplify a nucleic acid segment corresponding to a second region of the 18S rRNA gene.

In accordance with additional exemplary embodiments of the disclosure, a method of characterizing one or more protozoa includes the steps of preparing a nucleic acid library from a sample to form a plurality of nucleic acid segments having one or more of conserved and semi-conserved regions, sequencing the nucleic acid segments, and using a computer, characterizing the one or more protozoa based on the sequencing. The step of preparing a nucleic acid library from a sample can be performed using PCR.

In accordance with yet further exemplary embodiments of the disclosure, a method of characterizing one or more protozoa includes forming a plurality of nucleic acid segments having one or both of targeted conserved regions and targeted semi-conserved regions, characterizing the one or more protozoa based on the plurality of nucleic acid segments, and providing information about one or more of the identity, taxonomy, and relative contribution of the one or more protozoa in a sample. The method can include PCR. Further, the method can include a forward primer and a reverse primer to amplify segments of a first region of an 18S rRNA gene and/or a forward primer and a reverse primer to amplify segments of a second region of a 18S rRNA gene. The forward and/or reverse primers can include one or more degenerate bases and/or artificial or non-canonical bases.

In accordance with various embodiments, such as those set forth above, an exemplary forward primer for a first region of an 18S rRNA gene comprises CCATGCATGTCTAAGTATAAGC (SEQ ID NO: 1). An exemplary reverse primer for the first region of an 18S rRNA comprises CAGAAACTTGAATGATCTATCG (SEQ ID NO: 2). An exemplary forward primer for a second region of an 18S rRNA gene comprises RYGATYAGABACCVYYGTADTC (SEQ ID NO: 3). An exemplary reverse primer for the first region of an 18S rRNA comprises CGYGTTGAGTCRRATTR (SEQ ID NO: 4). In cases where the primers are not degenerate, the steps of generating or forming, e.g., using PCR amplification, can be and may desirably be performed under non-stringent conditions. In cases in which the primers include degenerate bases, the amplification may be done under stringent conditions. Exemplary primers can also include one or more of sequencing adapters, barcodes, and spacers. Using primers to amplify sections (e.g., conserved or semi-conserved regions) of the first region and the second region of the 18S rRNA gene allows for rapid characterization of protozoa within a sample.

Various methods described herein can be used to rapidly, e.g., within 12 hours or less, sequence protozoa nucleic acid—e.g., semi-conserved or conserved targeted nucleic acid regions of the first region and/or second region of the 18S rRNA gene—and characterize one or more protozoa present in a sample. Thus, the methods can be used in clinical applications where characterization of microorganisms, such as protozoa, is useful in the treatment of disease. By way of examples, use of primers to target first and second regions of the 18S rRNA gene can achieve the following. A) Exemplary primers are compatible with rapid DNA sequencing by functioning even with adapters (generally required by sequencing) and they yield amplicons that can be sequenced using existing methods (or rapid methods) and provide useful taxonomic information about the organisms in which they are derived. B) These primers are not only broad in scope, but provide useful results even in the background of clinical samples. Other primers are not be useful in the presence of human DNA. Exemplary primers were created and shown to work in the background of clinical samples. C) These two regions of the 18S rRNA gene taken together most protozoa.

Further, various methods can generate reports indicating one or more likely (characterized) protozoa present within a sample. The report can indicate a percent match to known protozoa, an amount of characterized protozoa present in the sample, taxonomic information of characterized protozoa, and/or indicated treatment for patients from which the analyzed sample was taken.

In accordance with further exemplary embodiments of the disclosure, a kit for characterizing microorganisms includes a forward primer comprising a priming sequence for a conserve or semi-conserve target sequence and a reverse primer. Exemplary forward and revers primers target first and/or second regions of the 18S rRNA gene.

Various additional embodiments of the disclosure relate to electronic systems and methods that can be used to characterize or identify one or more protozoa and optionally other microorganisms. For example, a method of characterizing one or more protozoa includes the step of selecting, by a computer, a digital file comprising one or more digital nucleic acid sequences (e.g., generated using a method described herein). The computer segments each of the one or more digital nucleic acid sequences into one or more first portions, performs a set of alignments by comparing the one or more first portions to information stored in a first database, and determines sequence portions from among the one or more first portions that have an alignment match (e.g., within a specified or predetermined range) to the information stored in the first database. Exemplary methods can further include performing, by the computer, a set of alignments by comparing the one or more first portions or one or more second portions to information stored in a second database or to information stored in the first database, determining sequence portions from among the one or more first portions or the one or more second portions that have an alignment match (e.g., within a specified or predetermined range) to the information stored in the first or second database, and characterizing one or more protozoa or nucleic acid fragments thereof based on the alignment match to the information stored in one or more of the first database and the second database. Exemplary methods can employ use of one, two, or more databases. Further, methods can include steps of comparing other information (other than sequences) to information in a database—e.g., the first, second, or another database. For example, an initial name attributed to a sequence could be compared to information stored in a database, and the name could be automatically changed based on such comparison. The modification could be, for example, to maintain information about one or more regions. In accordance with various aspects of these embodiments, the method can be used to characterize multiple microorganisms (e.g., including protozoa) simultaneously or in parallel, such that multiple microorganisms can be identified in a relatively short amount of time—e.g., preferably in less than forty-eight or less than twenty-four hours or less than 12 hours.

In accordance with further exemplary embodiments of the disclosure, an article of manufacture including a non-transitory computer readable medium having instructions stored thereon that, in response to execution by a computing device, causes the computing device to perform operations comprising the steps described in the above paragraph is provided.

In accordance with additional exemplary embodiments of the disclosure, a system includes a computer to perform one or more steps, such as the method steps noted above.

In accordance with further exemplary embodiments of the disclosure, a method of automatically characterizing one or more protozoa can be performed using one or more databases. Exemplary methods include the steps of detecting a sequence run that generates a digital nucleic acid sequence of one or more protozoa; selecting, by a computer, a digital file comprising one or more digital nucleic acid sequences, wherein each of the one or more digital nucleic acid sequences corresponds to a protozoa to be characterized; segmenting, by the computer, each of the one or more digital nucleic acid sequences into one or more portions; performing, by the computer, a set of alignments by comparing the one or more portions to information stored in one or more databases; determining, by the computer, sequence portions from among the one or more portions that have an alignment match to the information stored in the one or more databases; and characterizing one or more protozoa or nucleic acid fragments thereof based on the alignment match. In accordance with various aspects of these embodiments, the method can be used to characterize multiple microorganisms simultaneously, such that multiple microorganisms, including protozoa, can be identified in a relatively short amount of time—e.g., preferably in less than forty-eight or less than twenty-four hours or less than twelve hours.

Both the foregoing summary and the following detailed description are exemplary and explanatory only and are not restrictive of the present disclosure.

### BRIEF DESCRIPTION OF THE DRAWINGS

A more complete understanding of exemplary embodiments of the present disclosure may be derived by referring to the detailed description and claims when considered in connection with the following illustrative figures.

FIG. 1 illustrates genus level characterization of protozoa achieve using methods and kits in accordance with exemplary embodiments of the disclosure.

FIG. 2 illustrates species level characterization of protozoa achieve using methods and kits in accordance with exemplary embodiments of the disclosure.

FIG. 3 illustrates species and genus level characterization of protozoa achieve using methods and kits in accordance with exemplary embodiments of the disclosure

FIGS. 4-18 illustrate results obtained from multi-organism test assays in accordance with exemplary embodiments of the disclosure.

FIG. 19 illustrates results of a replica study using exemplary kits and methods in accordance with the disclosure.

FIG. 20 illustrates taxonomy of eukaryotic organisms detected using kits and methods in accordance with various embodiments of the disclosure.

It will be appreciated that elements in the figures are illustrated for simplicity and clarity and have not necessarily been drawn to scale. For example, the dimensions of some of the elements in the figures may be exaggerated relative to other elements to help to improve understanding of illustrated embodiments of the present disclosure.

### DETAILED DESCRIPTION

The description of exemplary embodiments of methods, assays, kits, and systems provided below is merely exemplary and is intended for purposes of illustration only; the following description is not intended to limit the scope of the disclosure or the claims. Moreover, recitation of multiple embodiments having stated features is not intended to exclude other embodiments having additional features or other embodiments incorporating different combinations of the stated features.

The following disclosure provides methods and kits for characterizing one or more protozoa. Various examples disclosed herein provide methods and kits for characterizing one or more protozoa or DNA fragments thereof, such as, pathogenic protozoa, in an efficient and timely manner, such that the systems and methods are suitable for use in clinical settings. Exemplary methods and kits can also provide information regarding the protozoa and/or information regarding treatment and/or treatment sensitivity information related to the one or more identified protozoa, such that a care provider can use such information.

As used herein, the term “subject” or “patient” refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal, such as a human.

Various embodiments of the present disclosure provide metagenomic or community profiling testing methods that use direct DNA sequencing and computational analysis to enable the detection, characterization or identification, and in the case of novel or divergent protozoa the identification of the nearest characterized protozoa species, and/or higher order taxonomic classification. Furthermore, exemplary methods can provide a relative measure of the protozoa contribution and diversity within a given sample. In these certain respects, the method may be called Pan-Protozoal Metagenomics or Pan-Protozoal Community Profiling as it aims to identify the genetic composition and diversity across multiple microorganisms in a sample, simultaneously.

Exemplary methods can characterize, identify, and/or survey the organisms including protozoa of an unknown or polymicrobial infection. By using direct DNA (nucleic acid) sequencing and computational analysis, these methods allow for the characterization or identification of the microorganisms. Adoption of the disclosed methods in clinical use can have far reaching implications not only by providing superior, unbiased, sequence based diagnosis, but also in reducing patient mortality, morbidity, length of stay, and associated hospital and healthcare costs. In accordance with some examples, ion semiconductor sequencing platforms or similar techniques are utilized to carry out the method because they enable an important aspect of this diagnostic method: speed. In certain aspects, the disclosed diagnostic method enables a turnaround time for results from a patient sample of about 12 hours, about 24 hours, about 48 hours, or about 72 hours or less. Exemplary methods can be performed as a Laboratory Developed Test (LDT) in a Clinical Laboratory Improvement Amendments (CLIA) regulated diagnostics laboratory. Various steps can be in accordance with CLIA, as set forth in U.S. patent application Ser. No. 14/196,999, filed Mar. 4, 2014, and entitled METHOD AND KIT FOR CHARACTERIZING MICROORGANISMS.

In accordance with various embodiments of the disclosure, a method of characterizing one or more microorganisms includes the steps of preparing an amplicon library with a polymerase chain reaction (PCR) of nucleic acids; sequencing a characteristic gene sequence in the amplicon library to obtain a gene sequence; and characterizing the one or more microorganisms (e.g., protozoa) based on the gene sequence using a computer-based genomic analysis of the gene sequence. The term “library”, as used herein refers to a library of organisms-derived nucleic acid sequences. The library may also have sequences allowing amplification of the library by the polymerase chain reaction or other in vitro amplification methods. The library may also have sequences that are compatible with next-generation high throughput sequencers.

In accordance with further exemplary embodiments of the disclosure, a method of characterizing one or more protozoa includes the steps of generating a plurality of nucleic acid segments from a sample using one or more degenerate primers to form a pool of nucleic acid segments having a target region, sequencing the pool of nucleic acid segments to form sequences including the target region, and using a computer, characterizing the one or more protozoa. The step of characterizing can include identifying the protozoa or identifying the nearest previously identified or know protozoa in a library. The characterizing or identifying can be based on taxonomic categories, such as species, genus, or higher order classifications of protozoa. In accordance with further aspects, the step of generating includes use of PCR, which can generate nucleic acid segments with one or more conserved regions and/or semi-conserved regions. By way of examples, forward and/or reverse primers with degenerate bases can be used with PCR to create a pool of nucleic acid segments that can be sequenced and analyzed, using a computer, to characterize the protozoa. Additionally or alternatively, one or more of the primer bases can be artificial or non-canonical. In accordance with further aspects of these embodiments, the step of generating includes amplifying a nucleic acid segment corresponding to a first region of an 18S rRNA gene. Exemplary methods can additionally or alternatively amplify a nucleic acid segment corresponding to a second region of an 18S rRNA gene.

In accordance with yet additional exemplary embodiments of the disclosure, a method of characterizing one or more protozoa includes the steps of preparing a nucleic acid library from a sample to form a plurality of nucleic acid segments having one or more of conserved and semi-conserved regions, sequencing the nucleic acid segments, and using a computer, characterizing the one or more protozoa based on the sequencing. The step of preparing a nucleic acid library from a sample can be performed using PCR.

In accordance with yet further exemplary embodiments of the disclosure, a method of characterizing one or more protozoa includes forming a plurality of nucleic acid segments having one or both of targeted conserved regions and targeted semi-conserved regions, characterizing the one or more protozoa based on the plurality of nucleic acid segments, and providing information about one or more of the identity, taxonomy, and relative contribution of the one or more protozoa in a sample. The method can include use of a forward primer and a reverse primer to amplify segments of a first region of an 18S rRNA gene and/or a forward primer and a reverse primer to amplify segments of a second region of a 18S rRNA gene. The forward and/or reverse primers can include one or more degenerate, artificial, and/or non-canonical bases. Further, the amplification of the segments from the first region or the second region can be performed under stringent or non-stringent conditions. By way of examples, non-degenerate primers can be used in connection with non-stringent conditions and primers having degenerate bases can be used in connection with stringent conditions.

In accordance with various embodiments set forth herein, an exemplary forward primer for a first region of an 18S rRNA gene comprises CCATGCATGTCTAAGTATAAGC (SEQ ID NO: 1). An exemplary reverse primer for the first region of an 18S rRNA comprises CAGAAACTTGAATGATCTATCG (SEQ ID NO: 2). An exemplary forward primer for a second region of an 18S rRNA gene comprises RYGATYAGABACCVYYGTADTC (SEQ ID NO: 3). An exemplary reverse primer for the first region of an 18S rRNA comprises CGYGTTGAGTCRRATTR (SEQ ID NO: 4). Exemplary primers can also include one or more of sequencing adapters, barcodes, and spacers, such as those described herein. Using primers to amplify sections (e.g., conserved or semi-conserved regions) of the first region and the second region of the 18S rRNA gene allows for rapid characterization of a wide range of protozoa within a sample.

In accordance with various aspects of the present disclosure, the target sequence is a segment from the 18S rRNA gene of a one or more protozoa. In some implementations, the target sequence may comprise material from a first region (region 1) and/or a second region (region 2) of the 18S rRNA gene. For example, first target material can comprise material from region 1 and second target material can comprise material from region 2 of the gene. The target sequence may be anywhere from about 5 nucleotides in length to about 40 nucleotides in length, from about 10 nucleotides in length to about 30 nucleotides in length, from about 15 nucleotides in length to about 25 nucleotides in length, or any suitable length.

Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U” denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage. Nucleotides may also be identified as indicated as shown below in Table 1.

TABLE 1
List of Nucleotide Abbreviations

| Symbol | Meaning | Origin of designation |
|---|---|---|
| A | A | adenine |
| G | G | guanine |
| C | C | cytosine |
| T | T | thymine |
| U | U | uracil |
| R | G or A | purine |
| Y | T/U or C | pyrimidine |
| M | A or C | amino |
| K | G or T/U | keto |
| S | G or C | strong interactions 3H- |
| | | bonds |
| W | A or T/U | weak interactions 2H- |
| | | bonds |
| B | G or C or T/U | not a |
| D | A or G or T/U | not c |
| H | A or C or T/U | not g |
| V | A or G or C | not t, not u |
| N | A or G or C or T/U, unknown, or other | any |

An artificial base and/or a non-canonical base can include: Inosine, Thiouridine, Uricil, Methyl-7-guanosine, Methylated RNA bases, RNA bases (if it were a hybrid molecule), Methylated DNA bases, Pseudouridine, Dihydrouridine, Dihydrouracil, Pseudouracil, Thiouracil, Methylcytosine, Methyl adenine, Isopentenyl adenine, Methyl guanidine, Queuosine, Wyosine, Diaminopurine, Isoguanine (isoC aka iso-dC), Isocytosine (isoG aka iso-dG), Diaminopyrimidine, Xanthine, Iosquinoline, Pyrrolo[2,3-b]pyridine, 2,4-difluorotoluene, 4-methylbenzimidazole, 2-amino-6-(2-thienyl)purine, pyrrole-2-carbaldehyde, 2,6-bis(ethylthiomethyl)pyridine (SPy and Ag ion), pyridine-2,6-dicarboxamide (Dipam), mondentate pyridine (Py) and Cu ion, 2′-deoxyinosine, Nitroazole-compounds, xDNA base pairs, yDNA base pairs, 2-amino-8-(2-thienyl)purine, pyridine-2-one, 7-(2-thienyl)imidazo[4,5-b]pyridine, pyrrole-2-carbaldehyde, 4-[3-(6-aminoheanamido)-1-propynyl]-2-nitropyrrole, 2-Aminopurine, 2,6-Diaminopurine (2-Amino-dA), 5-Bromo dU, deoxyuridine, Inverted dT, Inverted Dideoxy-T.

The term “pathogenic protozoa” as used herein refers to unicellular eukaryotic organisms that are known or suspected to contribute to human disease. Unless otherwise noted, “protozoa” can refer to a phylum, class, subclass, order, family, genus, species, or Glade of associated with the protozoa.

Chemically, a genome is composed of deoxy-ribonucleic acid (“DNA”). Each DNA molecule is made up of repeating units of four nucleotide bases—adenine (“A”), thymine (“T”), cytosine (“C”), and guanine (“G”)—which are covalently linked, or bonded, together via a sugar-phosphate, or phosphodiester, backbone. DNA generally exists as two DNA strands intertwined as a double helix in which each base on a strand pairs, or hybridizes, with a complementary base on the other strand: A pairs with T, and C with G.

The linear order of nucleotide bases in a DNA molecule is referred to as its “sequence.” The sequence of a gene is thus denoted by a linear sequence of As, Ts, Gs, and Cs. “DNA sequencing” or “gene sequencing” refers to the process by which the precise linear order of nucleotides in a DNA segment or gene is determined. A gene's nucleotide sequence in turn encodes for a linear sequence of amino acids that comprise the protein encoded by the gene. Most genes have both “exon” and “intron” sequences. Exons are DNA segments that are necessary for the creation of a protein, i.e., that code for a protein. Introns are segments of DNA.

Nearly every cell contains an entire genome. DNA in the cell, called “native” or “genomic” DNA, is packaged into chromosomes. Chromosomes are complex structures of a single DNA molecule wrapped around proteins called histones.

Genomic DNA can be extracted from its cellular environment using a number of well-established laboratory techniques. A particular segment of DNA, such as a gene, can then be excised or amplified from the DNA to obtain the isolated DNA segment of interest. DNA molecules can also be synthesized in the laboratory. One type of synthetic DNA molecule is complementary DNA (“cDNA”). cDNA is synthesized from mRNA using complementary base pairing in a manner analogous to RNA transcription. The process results in a double-stranded DNA molecule with a sequence corresponding to the sequence of an mRNA produced by the body. Because it is synthesized from mRNA, cDNA contains only the exon sequences, and thus none of the intron sequences, from a native gene sequence.

An oligonucleotide is a short segment of, e.g., RNA or DNA, typically comprising approximately thirty or fewer nucleotide bases. Oligonucleotides may be formed by the cleavage or division of longer RNA/DNA segments, or may by synthesized by polymerizing individual nucleotide precursors, such as by polymerase chain reaction (PCR) and/or other known techniques. Automated synthesis techniques such as PCR may allow the synthesis of oligonucleotides up to 10,000 or more nucleotide bases. With respect to PCR, an oligonucleotide is commonly referred to as a “primer,” which allows DNA polymerase to extend the oligonucleotide and replicate the complementary strand. The length of an oligonucleotide is typically denoted in terms of “mer.” By way of non-limiting example, an oligonucleotide having 25 nucleotide bases would be characterized as a 25-mer oligonucleotide. Because oligonucleotides readily bind to their respective complementary nucleotide, they may be used as probes for detecting particular DNA or RNA. The oligonucleotides can be made with standard molecular biology techniques known in the art and disclosed in manuals such as Sambrook et al., Molecular Cloning: A Laboratory Manual, Cold Spring Harbor Laboratory Press, Cold Spring Harbor, N.Y., USA (1989) or conventional nucleotide phosphoramidite chemistry and commercially available synthesizer instruments. The oligonucleotides can include DNA or RNA segments; also contemplated are the RNA equivalents of the oligonucleotides and their complements.

The term “primer” refers to an isolated single stranded oligonucleotide sequence capable of acting as a point of initiation for synthesis of a primer extension product, which is complementary to the nucleic acid strand to be copied. The length and the sequence of the primer are such that they can prime the synthesis of the extension products. A binding portion of a primer is generally about 5-50 nucleotides long, or from 10 to 40 nucleotides long. Specific length and sequence will depend on the complexity of the DNA or RNA targets, as well as on the conditions of primer use such as temperature and ionic strength.

As used herein, the terms “quantitative real time polymerase chain reaction,” “real-time polymerase chain reaction,” and “qPCR” are synonymous and refer to a laboratory technique based on a polymerase chain reaction used to amplify and simultaneously quantify a targeted DNA molecule. Frequently, real-time PCR is combined with reverse transcription to quantify messenger RNA and non-coding RNA in cells or tissues.

The oligonucleotides used as primers or probes may also comprise nucleotide analogues such as phosphorothiates, alkylphosphorothiates or peptide nucleic acids or may contain intercalating agents. As most other variations or modifications introduced into the original DNA sequences, these variations will necessitate adaptions with respect to the conditions under which the oligonucleotide should be used to obtain the desired specificity and sensitivity. However the eventual results of hybridization will be essentially the same as those obtained with the unmodified oligonucleotides. The introduction of these modifications may be advantageous in order to positively influence characteristics such as hybridization kinetics, reversibility of the hybrid-formation, biological stability of the oligonucleotide molecules, etc.

The term “sample” as used herein, means anything designated for testing for the presence of an organism and/or the nucleic acid of an organism. A sample is, or can be derived from any biological source, such as for example, blood, blood plasma, cell cultures, tissues and mosquito samples. The sample can be used directly as obtained from the source, or following a pre-treatment to modify the character of the sample. Thus, the sample can be pre-treated prior to use by, for example, preparing plasma from blood, disrupting cells or viral particles, preparing liquids from solid materials, diluting viscous fluids, filtering liquids, distilling liquids, concentrating liquids, inactivating interfering components, adding reagents, and purifying nucleic acid. A sample can include a clinical sample, such as a sample taken from blood, from the respiratory tract (sputum, bronchoalveolar lavage (BAL)), from cerebrospinal fluid (CSF), from the urogenital tract (vaginal secretions, urine), from the gastrointestinal tract (saliva, feces) or biopsies taken from organs, tissue, skin, teeth, bone, etc. A sample can also be an agricultural sample, such as a sample taken from soil, a plant, or an agricultural, waste water, sewage, or industrial process. The term sample can also refer to a sample of cultured cells, either cultured in liquid medium or on solid growth media. DNA present in said samples may be prepared or extracted according to any of the techniques known in the art. Exemplary techniques for extracting target nucleic acid are disclosed in U.S. patent application Ser. No. 13/834,441, filed Mar. 15, 2013, and entitled SEMI-PAN-PROTOZOAL BY QUANTITATIVE PCR, U.S. patent application Ser. No. 13/566,972, filed Aug. 3, 2012, and entitled COMPOSITIONS AND METHODS FOR DETECTING, EXTRACTING, VISUALIZING, AND IDENTIFYING PROTOMYXZOA RHUEMATIC, and U.S. patent application Ser. No. 14/331,143, filed Jul. 14, 2014, and entitled METHOD AND KIT FOR PROTOZOA CHARACTERIZATION.

The “target” material in these samples may be either segments of genomic DNA or precursor ribosomal RNA of the organism to be detected (target organism), or amplified versions thereof. These segment molecules are called target nucleic acids or target sequences.

A large number of protozoal pathogens are known. Exemplary methods and kits of the present disclosure may be used to detect a protozoan selected from the group consisting of Plasmodium, Protomyxzoa spp., Sarcocystis spp., Cyclophora spp., Eimeria spp., Goussia spp., Entomoeba histolytica, Acanthamoeba castellanii, Balamuthia mandrillaris, Trichomonas spp., Trypanosoma spp., Leishmania spp., Pneumocystis pneumonia, Naegleria fowleri, Giardia intestinalis, Blastocystis hominis, Babesia microti, Cryptosporidium spp., Cyclospora cayetanensis, Toxoplasma gondii, Theileria spp. The Protomyxzoa spp. may be Protomyxzoa rheumatica. The Cryptosporidium spp. may be Cryptosporidium parvum, Cryptosporidium hominis, Cryptosporidium canis, Cryptosporidium felis, Cryptosporidium meleagridis, or Cryptosporidium muris. The Trichomonas spp. may be Trichomonas tenas, Trichomonas hominis, or Trichomonas vaginalis. The Trypanosoma spp. may be Trypanosoma gambiense, Trypanosoma rhodesiense, Trypanosoma cruzi and Trypanosoma brucei. The Leishmania spp. may be Leishmania donovani, Leishmania tropica, or Leishmania braziliensis. The Theileria spp. may be Theileria lawrenci or Theileria parva.

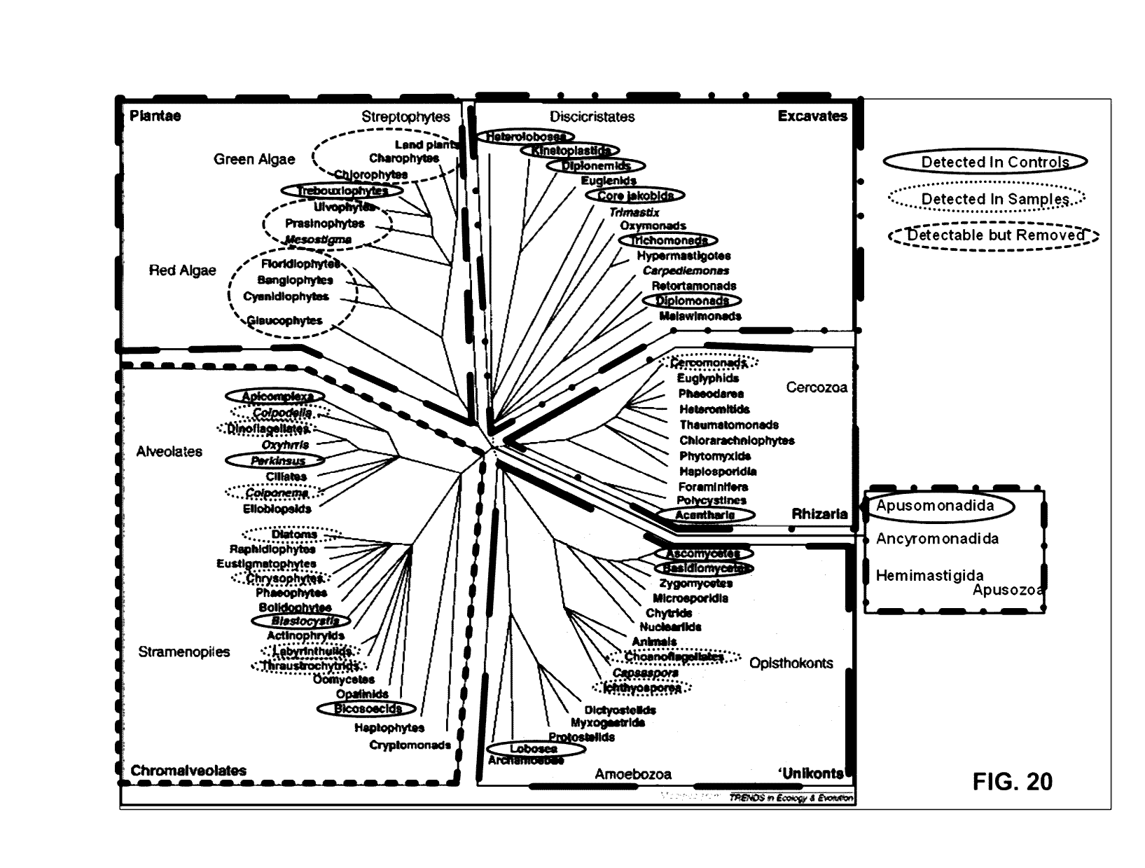

FIG. 20 illustrates taxonomy of eukaryotic organisms. Circles are placed around names of organisms within the illustrated groups that have been detected using the methods and kits described herein. The red circles correspond to standards that were prepared and tested; green circles correspond to organisms detected from actual samples sequenced by the laboratory; grey circles correspond to plants. The section that includes plants can be screened, except for the Trebouxiophytes because they contain actual human pathogens, so the actual capacity of the system likely includes all of the other plants as well.

In one aspect of the present disclosure, the pathogenic protozoa belongs to a phylum selected from the group consisting of Apicomplexa, Euglenozoa (e.g., Trypanosoma cruzi, Trypanosoma brucei, Leishmania spp.); Heterolobosea (e.g., Naegleria fowleri); Diplomonadida (e.g., Giardia intestinalis); Amoebozoa (e.g., Acanthamoeba castellanii, Balamuthia mandrillaris, Entamoeba histolytica); Blastocystis (e.g., Blastocystis hominis); Apicomplexa (e.g., Babesia microti, Cryptosporidium parvum, Cyclospora cayetanensis, Toxoplasma gondii). See Ecker D J, et al. (2005) “The Microbial Rosetta Stone Database: A compilation of global and emerging infectious microorganisms and bioterrorist threat agents” BMC Microbiol. 5:19.

The table below illustrates an exemplary compilation of the taxonomy of the standards used with exemplary embodiments of the disclosure.

| Species Name | Taxonomy |
|---|---|
| Acanthamoeba castellanii | Amoebozoa - Discosea |
| Adriamonas peritocrescens | Stramenopiles - Bigyra |
| Amastigomonas debruynei | Apusozoa - Apusomonadidae |
| Babesia microti | Alveolata - Apicomplexa |
| Blastocrithidia culicis | Euglenozoa - Kinetoplastida |
| Blastocystis hominis | Stramenopiles - Bigyra |
| Crithidia fasciculata | Euglenozoa - Kinetoplastida |
| Cryptococcus neoformans | Opisthokonta - Dikarya |
| Cryptosporidium parvum | Alveolata - Apicomplexa |
| Diplonema ambulator | Euglenozoa - Diplonemida |
| Endotrypanum monterogeii | Euglenozoa - Kinetoplastida |
| Entamoeba histolytica | Amoebozoa - Amoebozoa |
| Giardia intestinalis | Fornicata - Diplomonadida |
| Herpetomonas megaseliae | Euglenozoa - Kinetoplastida |
| Leishmania donovani | Euglenozoa - Kinetoplastida |
| Neospora caninum | Alveolata - Apicomplexa |
| Perkinsus marinus | Alveolata - Perkinsea |
| Plasmodium falciparum | Alveolata - Apicomplexa |
| Prototheca wickerhamii | Viridiplantae - Chlorophyta |
| Reclinomonas americana | Jakobida - Histionidae |
| Rhynchopus species | Euglenozoa - Diplonemida |
| Saccharomyces cerevisiae | Opisthokonta - Dikarya |
| Toxoplasma gondii | Alveolata - Apicomplexa |
| Trichomonas vaginalis | Parabasalia - Trichomonadida |
| Trypanosoma cruzi | Euglenozoa - Kinetoplastida |
| Vahlkampfia lobospinosa | Heterolobosea - Schizopyrenida |
| Wallaceina collosoma | Euglenozoa - Kinetoplastida |

There are many aspects of compositions, kits, and methods for detecting one or more protozoa disclosed herein, of which one, a plurality, or all aspects may be used in any particular implementation. It is to be understood that various implementations may be utilized, and, unless otherwise noted, compositional, as well as procedural, changes may be made without departing from the scope of this document. As a matter of convenience, various compositions and methods will be described using exemplary materials, sizes, specifications, and the like. However, this document is not limited to the stated examples and other configurations are possible and within the teachings of the present disclosure.

Implementations of the disclosed compositions, kits, and methods relate generally to oligonucleotides useful in methods for determining whether a sample contains one or more (e.g., pathogenic) protozoa and/or to characterizing the one or more protozoa. Any products such as peptides and the like are also within the scope of this disclosure. The detection and/or characterization of protozoa can be used as diagnostics for markers or in immunological testing as antigens.

In accordance with various embodiments of the disclosure, a forward primer and a reverse primer are configured to amplify one or more segments of a first region of the 18S rRNA gene of protozoa. The primers can include adapter sequence, a barcode sequence, a spacer, and/or other oligonucleotides used for priming a target sequence.

The term “barcode” or “barcode sequence” as used herein, refers to any unique, non-naturally occurring, nucleic acid sequence that may be used to identify the originating genome of a nucleic acid fragment. Barcodes may, optionally, be followed by a barcode adapter or spacer, for example, GAT. While exemplary barcodes are listed herein, any barcode of an appropriate length containing an arbitrary DNA sequence may be used with the method of the present disclosure. A length for the barcode may be about 5 nucleotides, about 6 nucleotides, about 7 nucleotides, about 8 nucleotides, about 9 nucleotides, about 10 nucleotides, about 15 nucleotides or about 20 nucleotides.

An “adapter sequence” is a nucleic acid that is generally not native to the target sequence, i.e. is exogenous, but is added or attached to the target sequence. The terms “barcodes,” “adapters,” “addresses,” “tags,” and “zip codes” have all been used to describe artificial sequences that are added to amplicons to allow separation of nucleic acid fragment pools. One exemplary form of adapters is hybridization adapters, which can be chosen so as to allow hybridization to the complementary capture probes on a surface of an array. Adapters serve as unique identifiers of the probe and thus of the target sequence. In general, sets of adapters and the corresponding capture probes on arrays are developed to minimize cross-hybridization with both each other and other components of the reaction mixtures, including the target sequences and sequences on the larger nucleic acid sequences outside of the target sequences (e.g. to sequences within genomic DNA). Other forms of adapters are mass tags that can be separated using mass spectroscopy, electrophoretic tags that can be separated based on electrophoretic mobility, etc. Some adapter sequences are outlined in U.S. Ser. No. 09/940,185, filed Aug. 27, 2001. Exemplary adapters are those that meet the following criteria. They are preferably not found in a genome, preferably a human or microbial genome, and they do not have undesirable structures, such as hairpin loops.

The attachment, or joining, of the adapter sequence to the target sequence can be done in a variety of ways. In one embodiment, the adapter sequences are added to the primers of the reaction (extension primers, amplification primers, readout probes, genotyping primers, Rolling Circle primers, etc.) during the chemical synthesis of the primers. The adapter then gets added to the reaction product during the reaction; for example, the primer gets extended using a polymerase to form the new target sequence that now contains an adapter sequence. Alternatively, the adapter sequences can be added enzymatically. Furthermore, the adapter can be attached to the target after synthesis; this post-synthesis attachment can be either covalent or non-covalent. In another embodiment the adapter is added to the target sequence or associated with a particular allele during an enzymatic step. That is, to achieve the level of specificity necessary for highly multiplexed reactions, the product of the specificity or allele specific reaction preferably also includes at least one adapter sequence. Additional adapter properties are described in U.S. patent application Ser. No. 14/196,999, filed Mar. 4, 2014, and entitled METHOD AND KIT FOR CHARACTERIZING MICROORGANISMS.

Exemplary primers suitable for priming the first region of this gene include the following.

Exemplary priming region of a forward primer for
region 1 of the 18S rRNA gene
(SEQ ID NO: 1)
CCATGCATGTCTAAGTATAAGC
Exemplary priming region of a reverse primer for
region 1 of the 18S rRNA gene
(SEQ ID NO: 2)
CAGAAACTTGAATGATCTATCG

The forward and reverse primers can additionally include a sequencing adapter sequence, a barcode sequence, and/or a spacer. Exemplary primers including an adapter sequence, a barcode sequence, and a spacer suitable for priming the first region of the 18S rRNA gene include the following, where reading the primers from left to right the first sequence portion is the sequencing adapter sequence. Next is the barcode sequence presented as bold and underlined followed by a spacer sequence (GAT) and lastly the colored primer binding sequence (shaded).

Exemplary forward primer for region 1 of the 18S rRNA gene

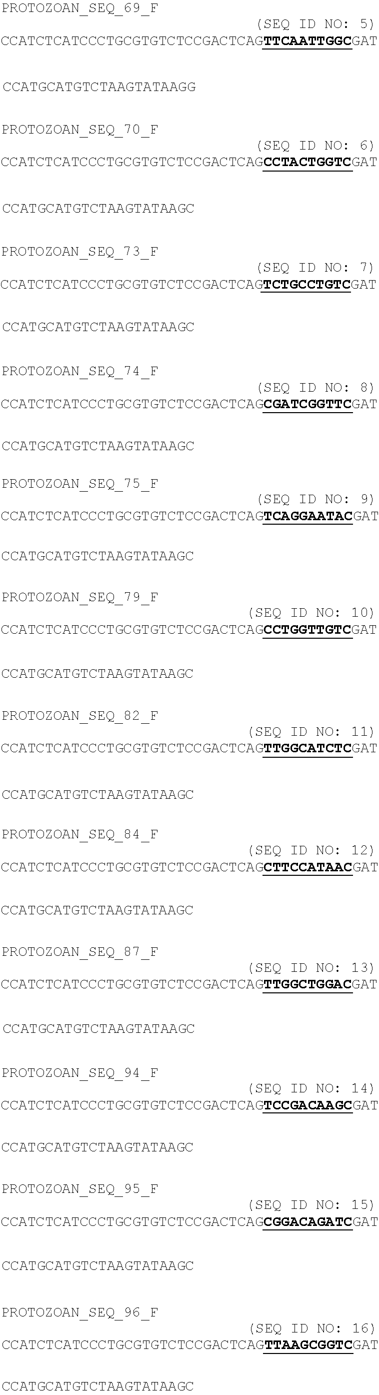

Exemplary reverse primer for region 1 of the 18S rRNA gene

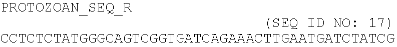

In accordance with further exemplary embodiments of the disclosure, a forward primer and a reverse primer are configured to amplify one or more segments of a second region of the 18S rRNA gene of protozoa. Exemplary primers suitable for priming the second region of the 18S rRNA gene include the following.

Exemplary priming region of a forward primer for
region 2 of the 18S rRNA gene
(SEQ ID NO: 3)
RYGATYAGABACCVYYGTADTC

This forward primer includes degenerate bases, which can generate 864 different priming regions.

Exemplary priming region of a reverse primer for
region 2 of the 18S rRNA gene
(SEQ ID NO: 4)
CGYGTTGAGTCRRATTR

This reverse primer includes degenerate bases, which can generate 16 different priming regions.

Similar to the primers described above, the primers for the second region can include a sequencing adapter sequence, a barcode sequence, and/or a spacer. Exemplary primers including an adapter sequence, a barcode sequence, and a spacer suitable for priming the first region of the 18S rRNA gene include the following, where reading the primers from left to right the first sequence portion is the sequencing adapter sequence. Next is the barcode sequence presented as bold and underlined followed by a spacer sequence (GAT) and lastly the shaded primer binding sequence. As noted above, the adapter sequence, a barcode sequence, and a spacer can include any suitable sequence. Further, the barcode can be variable (e.g., as illustrated below) and assigned to a sample or patient, such that multiple samples from, for example, multiple patients, can be run at the same time.

Exemplary forward primer for region 2 of the 18S rRNA gene

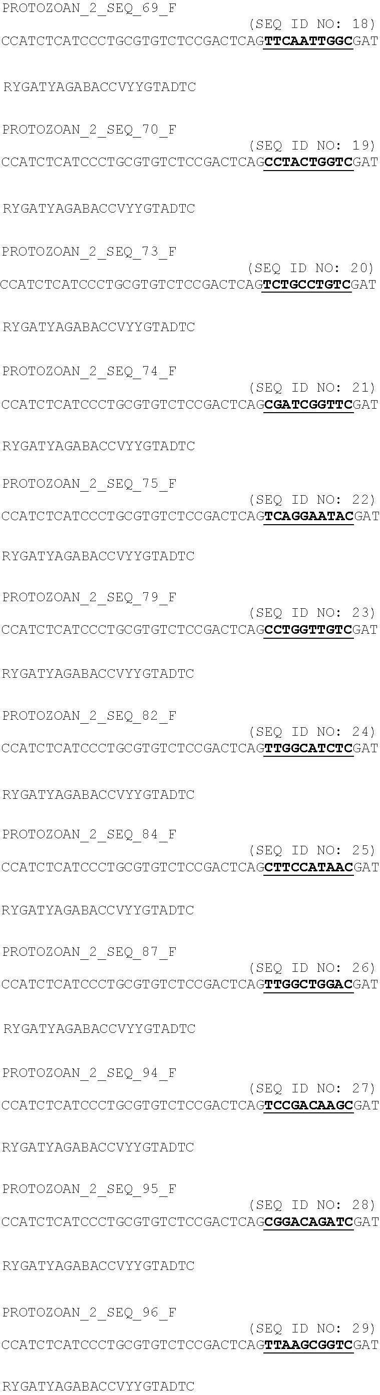

Exemplary reverse primer for region 2 of the 18S rRNA gene

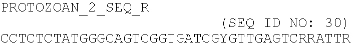

In accordance with various examples of these embodiments, the degenerate bases (position on sequences that can be more than one alternative base) can have about an equal probability of including one of the acceptable bases. In this context, the an amount of an acceptable base relative to another acceptable base varies by 1%, 2.5%, 5%, 10%, or 25%

In accordance with further examples, the one or more protozoa can be detected with qPCR utilizing any one of the following probes:

Pmyx_Clade_A_Probe1 (ROX)
(SEQ ID NO: 31)
/56-ROXN/GGATAACCGTAGTAATTCTGGAGCTAATACAT/
3IABRQSp/
Pmyx_Clade_B_Probel (HEX)
(SEQ ID NO: 32)
/HEX/TAAACTRTA/ZEN/ACTGWTWTAATGAGCYWTYCGCAGTTTY/
3IABkFQ/
Pmyx_Clade_C_Probe2 (Cy3)
(SEQ ID NO: 33)
/5Cy3/GGAGCTAATACATGATACAGGACCCG/3IAbRQSp/
Pmyx_Clade_D_Probe1 (Cy3)
(SEQ ID NO: 34)
/5Cy3/GAATGGCTCATTAWAWCAGTTAYAGTTTATTTGATGAT/
3IAbRQSp/
Pmyx_Clade_E_Probe1 (FAM)
(SEQ ID NO: 35)
/56-FAM/CTACGTGGATAACTGTAGTAATTCTAGAGCTAA/3IABkFQ/
Pmyx_Clade_E_Probe2 (FAM)
(SEQ ID NO: 36)
/56-FAM/TTATTTGAT/ZEN/GGTTTYYTACTTGGATAACCCGAGT/
3IABkFQ/
Pmyx_Clade_E_Probe3 (Cy5)
(SEQ ID NO: 37)
/5Cy5/CTCTGGCTAATATACGCTGAAGACC/3IAbRQSp/
Pmyx_Clade_F_Probe1 (Cy5)
(SEQ ID NO: 38)
/5Cy5/TGGATAACCGYRGTAATWCTRKRGCTAAKACATG/3IAbRQSp/
Pmyx_Clade_G_Probe1 (Cy5)
(SEQ ID NO: 39)
/5Cy5/GTGAAACTGCGAATGGCTCATTATATCAGTTAT/3IAbRQSp/
Pmyx_Clade_H_Probe1 (FAM)
(SEQ ID NO: 40)
/56-FAM/WAYDGYGAA/ZEN/ACTGCGAATGGCTCATTAWAWCA/
3IABkFQ/
FL1953_Probe (FAM)
(SEQ ID NO: 41)
FAM-ACATCCTTT/ZEN/CCGTGAGGTCAGGAGTT-3IABkFQ

In another aspect, implementations of the disclosed compositions and methods relate generally to oligonucleotides, recombinant products such as peptides, and the like useful in methods for determining whether a sample contains one or more protozoa, or has an increased likelihood of containing one or more protozoa. Protozoa has been associated with diseases, such as CFS, Fibromyalgia, the autoimmune diseases, ALS, MS, Parkinson's disease, Autism, Toxoplasmosis, Acanthamoebiasis, Malaria, Babesiosis, Trypanosomiasis, Leshmaniasis, and the like. Therefore detection and/or characterization of protozoa can be helpful in diagnosis and/or treatment of such diseases.

In yet another aspect, methods useful for detecting one or more protozoa from one or more samples may comprise aligning nucleotide sequences pair wise and determining the percent identities (percentage of identical matches) between universal and/or specific primers and the sample to be tested. In particular implementations, a reaction mixture or a kit may be provided comprising an isolated oligonucleotide (a forward primer, in particular implementations). In other particular implementations, a second isolated oligonucleotide, different than the first isolated oligonucleotide (a reverse primer, in particular implementations) may be provided. The primers are capable of hybridizing under highly stringent hybridization conditions to a polynucleotide present in the sample. In accordance with yet further aspects and by way of examples, the kits include forward and reverse primers for priming a first region of an a 18S rRNA gene and/or forward and reverse primers for priming a second region of the 18S rRNA gene. Including forward and reverse primers for both regions of the 18S rRNA gene allows characterization of a wide array of protozoa, such as those noted herein.

Methods useful for detecting one or more protozoa from one or more samples may further comprise a method for determining whether a sample contains one or more protozoa or has an increased likelihood of containing one or more protozoa, wherein the method includes:
a) providing a vessel containing a reaction mixture, wherein the reaction mixture comprises at least one forward primer as described herein, at least one reverse primer as described herein, and a nucleic acid target from the sample; wherein the reaction mixture is capable of amplifying, by a polymerase chain reaction (PCR), a segment of the nucleic acid target to produce an amplicon; and wherein production of the amplicon is primed by the at least one forward primer and the at least one reverse primer;
b) incubating the vessel under conditions allowing production of the amplicon if the sample contains one or more protozoa; and
c) determining that the sample contains one or more protozoa or that the sample has an increased likelihood of containing the one or more protozoa if the amplicon is detected, or determining that the sample does not contain the pathogenic protozoan or that the sample does not have an increased likelihood of containing the pathogenic protozoan if the amplicon is not detected.

Alternatively, in step a) of the method above, the reaction mixture may further comprise an oligonucleotide probe (by way of non-limiting example, a molecular beacon) capable of detecting the amplicon if the amplicon is produced.

Nucleic acids, including oligonucleotide probes, in the methods and compositions described herein may be labeled with a reporter. A reporter is a molecule that facilitates the detection of a molecule to which it is attached. Numerous reporter molecules that may be used to label nucleic acids are known. Direct reporter molecules include fluorophores, chromophores, and radiophores. Non-limiting examples of fluorophores include, a red fluorescent squarine dye such as 2,4-Bis[1,3,3-trimethyl-2-indolinylidenemethyl]cyclobutenediylium-1,3-dioxolate, an infrared dye such as 2,4-Bis[3,3-dimethyl-2-(1H-benz[e]indolinylidenemethyl)]cyclobutenediylium-1,3-dioxolate, or an orange fluorescent squarine dye such as 2,4-Bis[3,5-dimethyl-2-pyrrolyl]cyclobutenediylium-1,3-diololate. Additional non-limiting examples of fluorophores include quantum dots, Alexa Fluor® dyes, AMCA, BODIPY® 630/650, BODIPY® 650/665, BODIPY®-FL, BODIPY®-R6G, BODIPY®-TMR, BODIPY® TRX, Cascade Blue®, CyDye™, including but not limited to Cy2™, Cy3™, and Cy5™, a DNA intercalating dye, 6-FAM™, Fluorescein, HEX™, 6-JOE, Oregon Green® 488, Oregon Green® 500, Oregon Green® 514, Pacific Blue™, REG, phycobilliproteins including, but not limited to, phycoerythrin and allophycocyanin, Rhodamine Green™, Rhodamine Red™, ROX™, TAMRA™, TET™, Tetramethylrhodamine, or Texas Red®. A signal amplification reagent, such as tyramide (PerkinElmer), may be used to enhance the fluorescence signal. Indirect reporter molecules include biotin, which must be bound to another molecule such as streptavidin-phycoerythrin for detection. In a multiplex reaction, the reporter attached to the primer or the dNTP may be the same for all reactions in the multiplex reaction if the identities of the amplification products can be determined based on the specific location or identity of the solid support to which they hybridize.

It is also contemplated that fluorophore/quencher-based detection systems may be used with the methods and compositions disclosed herein. When a quencher and fluorophore are in proximity to each other, the quencher quenches the signal produced by the fluorophore. A conformational change in the nucleic acid molecule separates the fluorophore and quencher to allow the fluorophore to emit a fluorescent signal. Fluorophore/quencher-based detection systems reduce background and therefore allow for higher multiplexing of primer sets compared to free floating fluorophore methods, particularly in closed tube and real-time detection systems.

In particular embodiments, molecules useful as quenchers include, but are not limited to tetramethylrhodamine (TAMRA), DABCYL (DABSYL, DABMI or methyl red) anthroquinone, nitrothiazole, nitroimidazole, malachite green, Black Hole Quenchers®, e.g., BHQ1 (Biosearch Technologies), Iowa Black® or ZEN quenchers (from Integrated DNA Technologies, Inc.) (e.g., 3′ Iowa Black® RQ-Sp aka 3IABRQSp and 3′ Iowa Black® FQ aka 3IABkFQ), TIDE Quencher 2 (TQ2) and TIDE Quencher 3 (TQ3) (from AAT Bioquest).

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.); Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like. Commercially available linking moieties can be employed that can be attached to an oligonucleotide during synthesis, e.g., available from Integrated DNA Technologies (Coralville, Iowa) or Eurofins MWG Operon (Huntsville, Ala.).

Amplifying or generating steps as described herein can be performed using any type of nucleic acid template-based method, such as PCR technology. PCR is a technique widely used in molecular biology to amplify a piece of DNA by in vitro enzymatic replication. Typically, PCR applications employ a heat-stable DNA polymerase, such as Taq polymerase. This DNA polymerase enzymatically assembles a new DNA strand from nucleotides (dNTPs) using single-stranded DNA as template and DNA primers to initiate DNA synthesis. A basic PCR reaction uses several components and reagents including: a DNA template that contains the target sequence to be amplified; one or more primers, which are complementary to the DNA regions at the 5′ and 3′ ends of the target sequence; a DNA polymerase (e.g., Taq polymerase) that preferably has a temperature optimum at around 70° C.; deoxynucleotide triphosphates (dNTPs); a buffer solution providing a suitable chemical environment for optimum activity and stability of the DNA polymerase; divalent cations, typically magnesium ions (Mg2+); and monovalent cation potassium ions.

PCR technology uses thermal strand separation followed by thermal dissociation. During this process, at least one primer per strand, cycling equipment, high reaction temperatures and specific thermostable enzymes are used (See, e.g., U.S. Pat. Nos. 4,683,195 and 4,883,202). Alternatively, it is possible to amplify the nucleic acid at a constant temperature (Nucleic Acids Sequence Based Amplification (NASBA) Kievits, T., et al., J. Virol Methods, 1991; 35, 273-286; and Malek, L. T., U.S. Pat. No. 5,130,238; T7 RNA polymerase-mediated amplification (TMA) (Giachetti C, et al., J Clin Microbiol 2002 July; 40(7):2408-19; or Strand Displacement Amplification (SDA), Walker, G. T. and Schram, J. L., European Patent Application Publication No. 0 500 224 A2; Walker, G. T., et al., Nuc. Acids Res., 1992; 20, 1691-1696).

Thermal cycling subjects the PCR sample to a defined series of temperature steps. Each cycle typically has 2 or 3 discrete temperature steps. The cycling is often preceded by a single temperature step (“initiation”) at a high temperature (>90° C.), and followed by one or two temperature steps at the end for final product extension (“final extension”) or brief storage (“final hold”). The temperatures used and the length of time they are applied in each cycle depend on a variety of parameters. These include the enzyme used for DNA synthesis, the concentration of divalent ions and dNTPs in the reaction, and the melting temperature (Tm) of the primers. Commonly used temperatures for the various steps in PCR methods are: initialization step—94-96° C.; denaturation step—94-98° C.; annealing step—50-65° C.; extension/elongation step—70-74° C.; final elongation—70-74° C.; final hold—4-10° C.

As noted above, qPCR can be used to amplify and simultaneously quantify target nucleic acid(s). qPCR enables both detection and quantification (as absolute number of copies or relative amount when normalized to DNA input or additional normalizing genes) of a specific sequence in a DNA sample. Real-time PCR may be combined with reverse transcription polymerase chain reaction to quantify low abundance RNAs. Relative concentrations of DNA present during the exponential phase of real-time PCR are determined by plotting fluorescence against cycle number on a logarithmic scale. Amounts of DNA may then be determined by comparing the results to a standard curve produced by real-time PCR of serial dilutions of a known amount of DNA.

Multiplex-PCR and multiplex real-time PCR use of multiple, unique primer sets within a single PCR reaction to produce amplicons of different DNA sequences. By targeting multiple genes at once, additional information may be gained from a single test run that otherwise would require several times the reagents and more time to perform. Annealing temperatures for each of the primer sets should be optimized to work within a single reaction.

Multiplex-PCR and multiplex real-time PCR may also use unique sets or pools of oligonucleotide probes to detect multiple amplicons at once. In some embodiments, the method of the present invention comprises multiplex quantitative real time PCR (qPCR) with unique pools of oligonucleotide probes. In one embodiment, the reaction mixture in the multiplex qPCR comprises a pool of oligonucleotide probes selected from:

(a) SEQ ID NO: 31, SEQ ID NO: 32, and SEQ ID NO: 41;

(b) SEQ ID NO: 33, SEQ ID NO: 35, and SEQ ID NO: 38;

(c) SEQ ID NO: 34, SEQ ID NO: 36, and SEQ ID NO: 37; and

(d) SEQ ID NO: 39, and SEQ ID NO: 40.

The methods disclosed herein may also utilize asymmetric priming techniques during the PCR process, which may enhance the binding of the reporter probes to complimentary target sequences. Asymmetric PCR is carried with an excess of the primer for the chosen strand to preferentially amplify one strand of the DNA template more than the other.

Amplified nucleic acid can be detected using a variety of detection technologies well known in the art. For example, amplification products may be detected using agarose gel by performing electrophoresis with visualization by ethidium bromide staining and exposure to ultraviolet (UV) light, by sequence analysis of the amplification product for confirmation, or hybridization with an oligonucleotide probe.

The oligonucleotide probe may comprise a flourophore and/or a quencher. The oligonucleotide probe may also contain a detectable label including any molecule or moiety having a property or characteristic that is capable of detection, such as, for example, radioisotopes, fluorophores, chemiluminophores, enzymes, colloidal particles, and fluorescent microparticles.

Probe sequences can be employed using a variety of methodologies to detect amplification products. Generally the methods employ a step where the probe hybridizes to a strand of an amplification product to form an amplification product/probe hybrid. The hybrid can then be detected using, e.g., labels on the primer, probe or both the primer and probe. Examples of homogeneous detection platforms for detecting amplification products include the use of FRET (fluorescence resonance energy transfer) labels attached to probes that emit a signal in the presence of the target sequence. “TaqMan” assays described in U.S. Pat. Nos. 5,210,015; 5,804,375; 5,487,792 and 6,214,979 and Molecular Beacon assays described in U.S. Pat. No. 5,925,517 are examples of techniques that can be employed to detect nucleic acid sequences. With the “TaqMan” assay format, products of the amplification reaction can be detected as they are formed or in a so-called “real time” manner. As a result, amplification product/probe hybrids are formed and detected while the reaction mixture is under amplification conditions.

For example, the PCR probes may be TaqMan® probes that are labeled at the 5′ end with a fluorophore and at the 3′-end with a quencher molecule. Suitable fluorophores and quenchers for use with TaqMan® probes are disclosed in U.S. Pat. Nos. 5,210,015, 5,804,375, 5,487,792 and 6,214,979 and WO 01/86001 (Biosearch Technologies). Quenchers may be Black Hole Quenchers disclosed in WO 01/86001.

Nucleic acid hybridization can be done using techniques and conditions known in the art. Specific hybridization conditions will depend on the type of assay in which hybridization is used. Hybridization techniques and conditions can be found, for example, in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York) and Sambrook et al. (1989) Molecular Cloning. A Laboratory Manual (2d ed., Cold Spring Harbor Laboratory Press, Plainview, N.Y.).

Hybridization of nucleic acid may be carried out under stringent conditions. “Stringent conditions” or “stringent hybridization conditions” can mean conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified. Alternatively, stringency conditions can be adjusted to allow some mismatching in sequences so that lower degrees of similarity are detected. For example, as noted above, when non-stringent conditions are desired—e.g., when using a primer that does not include degenerate bases—e.g., in the priming region, the annealing temperature may be about five degrees or more less than Tm. Conversely, when primers include degenerate bases in the priming region the annealing temperature can be about five degrees or less of Tm.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1.times. to 2.times.SSC (20.times.SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5.times. to 1.times.SSC at 55 to 60° C. Exemplary high stringency conditions include hybridization in 50% formamide, 1 M NaCl, 1% SDS at 37° C., and a wash in 0.1.times.SSC at 60 to 65° C. The duration of hybridization is generally less than about 24 hours, usually about 4 to about 12 hours, or less depending on the assay format.

It should be noted that the oligonucleotides of this disclosure can be used as primers or probes, depending on the intended use or assay format. For example, an oligonucleotide used as a primer in one assay can be used as a probe in another assay. The grouping of the oligonucleotides into primer pairs and primer/probe sets reflects certain implementations only. However, the use of other primer pairs comprised of forward and reverse primers selected from different preferred primer pairs is specifically contemplated.

Exemplary sample and library preparation in accordance with various examples includes:

1. DNA Extraction

2. Amplification and Barcoding

3. DNA Purification

4. IonSphere Particle Labeling

5. IonSphere Particle Enrichment

DNA extraction may be accomplished by any method available in the art. Nucleic acids can be extracted from a biological sample by a variety of techniques such as those described by Maniatis, et al., Molecular Cloning: A Laboratory Manual, Cold Spring Harbor, N.Y., pp. 280-281, (1982). In one embodiment, DNA is extracted from the biological sample with the QIAamp® DNA Mini Kit.

Sample and Library Preparation may also involve the running of a polymerase chain reaction (PCR). As noted above, PCR is a technique in molecular biology to amplify a single or few copies of a piece of DNA across several orders of magnitude, generating thousands to millions of copies of a particular DNA sequence. The method relies on thermal cycling, consisting of cycles of repeated heating and cooling of the reaction for DNA melting and enzymatic replication of the DNA. Primers (short DNA fragments) containing sequences complementary to the target region along with a DNA polymerase (after which the method is named) are key components to enable selective and repeated amplification. As PCR progresses, the DNA generated is itself used as a template for replication, setting in motion a chain reaction in which the DNA template is exponentially amplified. PCR can be extensively modified to perform a wide array of genetic manipulations.

Many PCR applications employ a heat-stable DNA polymerase, such as Taq polymerase, an enzyme originally isolated from the bacterium Thermus aquaticus. This DNA polymerase enzymatically assembles a new DNA strand from DNA building blocks, the nucleotides, by using single-stranded DNA as a template and DNA oligonucleotides (also called DNA primers), which are required for initiation of DNA synthesis. The vast majority of PCR methods use thermal cycling, i.e., alternately heating and cooling the PCR sample to a defined series of temperature steps. These thermal cycling steps are used first to physically separate the two strands in a DNA double helix at a high temperature in a process called DNA melting. At a lower temperature, each strand is then used as the template in DNA synthesis by the DNA polymerase to selectively amplify the target DNA. The selectivity of PCR results from the use of primers that are complementary to the DNA region targeted for amplification under specific thermal cycling conditions. In one embodiment, the present disclosure contemplates a method comprising amplifying a plurality of a complex mixture (“library”) of DNA molecules by PCR.

PCR is used to amplify a specific region of a DNA strand (the target material). Most PCR methods typically amplify DNA fragments of up to ˜10 kilo base pairs (kb), although some techniques allow for amplification of fragments up to 40 kb in size. Cheng et al., “Effective amplification of long targets from cloned inserts and human genomic DNA” Proc Natl Acad Sci. 91: 5695-5699 (1994). A basic PCR set up usually involves several components and reagents. “Chapter 8: In vitro Amplification of DNA by the Polymerase Chain Reaction” In: Molecular Cloning: A Laboratory Manual (3rd ed.) Sambrook et al. (Eds). Cold Spring Harbor, N.Y.: Cold Spring Harbor Laboratory Press. ISBN 0-87969-576-5 (2001). These components may include, but are not limited to: i) DNA template that contains the DNA region (target) to be amplified; ii) two primers that are complementary to the 3′ ends of each of the sense and anti-sense strand of the DNA target; iii) Taq polymerase or another DNA polymerase with a temperature optimum at around 70° C.; iv) deoxynucleoside triphosphates (dNTPs; also very commonly and erroneously called deoxynucleotide triphosphates), the building blocks from which the DNA polymerases synthesizes a new DNA strand; v) buffer solution, providing a suitable chemical environment for optimum activity and stability of the DNA polymerase; vi) divalent cations, magnesium or manganese ions; generally Mg2+ is used, but Mn2+ can be utilized for PCR-mediated DNA mutagenesis, as higher Mn2+ concentration increases the error rate during DNA synthesis (Pavlov et al., “Recent developments in the optimization of thermostable DNA polymerases for efficient applications” Trends Biotechnol. 22: 253-260 (2004)); and vii) monovalent cation potassium ions.

The PCR is commonly carried out in a reaction volume of 10-200 μl in small reaction tubes (0.2-0.5 ml volumes) in a thermal cycler. The thermal cycler heats and cools the reaction tubes to achieve the temperatures required at each step of the reaction. Many modern thermal cyclers make use of the Peltier effect which permits both heating and cooling of the block holding the PCR tubes simply by reversing the electric current. Thin-walled reaction tubes permit favorable thermal conductivity to allow for rapid thermal equilibration. Most thermal cyclers have heated lids to prevent condensation at the top of the reaction tube, but a layer of oil or a ball of wax may also be effective.

In some embodiments, the method of the present disclosure comprises preparing an ion amplicon library. This may be accomplished with the fusion PCR method using fusion primers to attach the Ion A and truncated P1 (trP1) Adapters to the amplicons as they are generated in PCR. The fusion primers contain the A and trP1 sequences at their 5′-ends adjacent to the target-specific portions of the primers. The target region is the portion of the genome that will be sequenced in the samples of interest. For example the target region could be an exon, a portion of an exon, or a non-coding region of the genome. Primers are designed so that any sequence variants of interest are located between the primers and so those variants are not masked by the template-specific part of the primer sequences. The length of the target region is also carefully considered. In one embodiment, bidirectional sequencing is used. In another embodiment, sequencing proceeds in a single direction.

For bidirectional sequencing, the fusion PCR method for preparing an amplicon library generally requires four fusion primers: two pairs of forward and reverse primers per target region. If sequencing proceeds in a single direction, only one pair of forward and reverse primers per target is required. The amplicons are designed so that their length, including the fusion primers with adapter sequences, is shorter than the median library size for the target read length of the library.

Design of Amplicon Length

| Target Read Length | Median Library Size |
|---|---|
| 200 bases (200 base-read library) | ~330 bp |
| 100 bases (100 base-read library) | ~200 bp |

One fusion primer pair has the A adapter region followed by the proximal end of the target sequence, and the other has the trP1 adapter region followed by the distal end of the target sequence. The other fusion primer pair has the adapter sequences A and trP1 swapped. The target-specific portion of each primer should include 15-20 nucleotides of the target region.

In some aspects of the present disclosure, sequencing proceeds in one direction and the reverse primers do not include a barcode sequence or a barcode adapter.

In some embodiments, Ion Semiconductor Sequencing is utilized to analyze the purified DNA from the sample. Ion Semiconductor Sequencing is a method of DNA sequencing based on the detection of hydrogen ions that are released during DNA amplification. This is a method of “sequencing by synthesis,” during which a complementary strand is built based on the sequence of a template strand.

For example, a microwell containing a template DNA strand to be sequenced can be flooded with a single species of deoxyribonucleotide (dNTP). If the introduced dNTP is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple dNTP molecules will be incorporated in a single cycle. This leads to a corresponding number of released hydrogens and a proportionally higher electronic signal.

This technology differs from other sequencing technologies in that no modified nucleotides or optics are used. Ion semiconductor sequencing may also be referred to as ion torrent sequencing, pH-mediated sequencing, silicon sequencing, or semiconductor sequencing. Ion semiconductor sequencing was developed by Ion Torrent Systems Inc. and may be performed using a bench top machine. Rusk, N. (2011). “Torrents of Sequence,” Nat Meth 8(1): 44-44. Although it is not necessary to understand the mechanism of an invention, it is believed that hydrogen ion release occurs during nucleic acid amplification because of the formation of a covalent bond and the release of pyrophosphate and a charged hydrogen ion. Ion semiconductor sequencing exploits these facts by determining if a hydrogen ion is released upon providing a single species of dNTP to the reaction.

For example, microwells on a semiconductor chip that each contain one single-stranded template DNA molecule to be sequenced and one DNA polymerase can be sequentially flooded with unmodified A, C, G or T dNTP. Pennisi, E. (2010). “Semiconductors inspire new sequencing technologies” Science 327(5970): 1190; and Perkel, J., “Making contact with sequencing's fourth generation” Biotechniques (2011). The hydrogen ion that is released in the reaction changes the pH of the solution, which is detected by a hypersensitive ion sensor. The unattached dNTP molecules are washed out before the next cycle when a different dNTP species is introduced.

Beneath the layer of microwells is an ion sensitive layer, below which is a hypersensitive ISFET ion sensor. All layers are contained within a CMOS semiconductor chip, similar to that used in the electronics industry. Each released hydrogen ion triggers the ISFET ion sensor. The series of electrical pulses transmitted from the chip to a computer is translated into a DNA sequence, with no intermediate signal conversion required. Each chip contains an array of microwells with corresponding ISFET detectors. Because nucleotide incorporation events are measured directly by electronics, the use of labeled nucleotides and optical measurements are avoided.

An example of a Ion Semiconductor Sequencing technique suitable for use in the methods of the provided disclosure is Ion Torrent sequencing (U.S. Patent Application Numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (H⁺), which signal detected and recorded in a sequencing instrument. The signal strength is proportional to the number of nucleotides incorporated. User guides describe in detail the Ion Torrent protocol(s) that are suitable for use in methods of the invention, such as Life Technologies' literature entitled “Ion Sequencing Kit for User Guide v. 2.0” for use with their sequencing platform the Personal Genome Machine™ (PCG).

Kits according to the disclosure include one or more reagents useful for practicing one or more assay methods of the disclosure. A kit generally includes a package with one or more containers holding the reagent(s) (e.g., primers and/or probe(s) as described herein), as one or more separate compositions or, optionally, as admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Exemplary kits include at least one oligonucleotide (e.g., forward or reverse primer) disclosed in this document. The kits may contain one or more pairs of oligonucleotides such as the primer pairs disclosed herein, or one or more oligonucleotide sets as disclosed herein. The kit can further comprise the fourdeoxynucleotide phosphates (dATP, dGTP, dCTP, dTTP) and an effective amount of a nucleic acid polymerizing enzyme. A number of enzymes are known in the art which are useful as polymerizing agents. These include, but are not limited to E. coli DNA polymerase I, Klenow fragment, bacteriophage T7 RNA polymerase, reverse transcriptase, and polymerases derived from thermophilic bacteria, such as Thermus aquaticus. The latter polymerases are known for their high temperature stability, and include, for example, the Taq DNA polymerase I. Other enzymes such as Ribonuclease H can be included in the kit for regenerating the template DNA. Other optional additional components of the kit include, for example, means used to label the probe and/or primer (such as a fluorophore, quencher, chromogen, etc.), and the appropriate buffers for reverse transcription, PCR, or hybridization reactions.

Kits according to the disclosure can also include instructions for carrying out one or more of the methods of the disclosure. Instructions included in kits of the disclosure can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), RF tags, and the like. As used herein, the term “instructions” can include the address of an internet site that provides the instructions.

The following references may provide exemplary procedural or other details supplementary to those set forth herein. Bengtsson et al., Nucleic Acids Res., 31:e45, 2003. Bernard et al., Am. J. Pathol., 153:1055-1061, 1998. Bernard et al., Anal. Biochem., 255:101-107, 1998. Bustin et al., J. Biomol. Tech., 15:155-166, 2004. Bustin, J. Mol. Endocrinol., 29(1):23-39, 2002. Cardullo et al., Proc. Natl. Acad. Sci. USA, 85:8790-8804, 1988. Chen et al., J. Virol. Methods, 122(1):57-61, 2004. Dorak, In: Real-time PCR, Bios Advanced Methods, 1st Ed., Taylor & Francis, 2006. Egholm et al., Nature, 365(6446):566-568, 1993. Espy et al., Clin. Microbiol. Rev., 19(1):165-256, 2006. Guo et al., Nat. Biotechnol., 4:331-335, 1997. Higuchi et al., Biotechnol., 10: 412-417, 1992. Higuchi et al., Biotechnol., 11:1026-1030, 1993. Ishiguro et al., Anal. Biochemistry, 229(2): 207-213, 1995. Johnson et al., Nucl. Acids Res., 32:1937-1941, 2004. Koshkin and Dunford, J. Biol. Chem., 273(11):6046-6049, 1998a. Koshkin and Wengel, J. Org. Chem., 63(8):2778-2781, 1998b. Lay and Wittwer, Clin. Chem., 1997; 43: 2262-2267, 1997. Morrison et al., Anal. Biochem., 183:231-244, 1989. Morrison et al., Biochemistry, 32:3095-3104, 1993. Moser et al., Nucl. Acids Res., 31:5048-5053, 2003. Mueller et al., Current Protocols in Mol. Biol.; 15:5, 1993. Nazarenko et al., Nucleic Acids Res., 25(12):2516-2521, 1997. Nazarenko et al., Nucleic Acids Res., 30(9):37, 2002. Nygren et al., Biopolymers, 46:39-51, 1998. Sano, T. et al., Science, 258:120-122, 1992. Santalucia et al., Biochemistry; 38:3468-3477, 1999. Sherrill et al., J. Am. Chem. Soc., 126:4550-4556, 2004. Sims, P W et al., Anal Biochem. 281:230-232, 2000. Wahlestedt et al., Proc. Natl. Acad. Sci. USA, 97(10):5633-5638, 2000. Whitcombe et al., Nat. Biotechnol., 17:804-807, 1999. Wilhelm and Pingoud, Chem. BioChem., 4:1120-1128, 2003. Wittwer et al., Biotechniques, 22:130-138, 1997. Zipper et al., Nucleic Acids Res., 32(12):103, 2004.

As noted above, some exemplary methods of the present disclosure comprises characterizing—e.g., classifying the species or genus or other taxonomic classification of one or more protozoa with a computer-based genomic analysis of the sequence data from, for example, an ion semiconductor sequencing platform or other suitable (e.g., next generation) platform. The methods may further comprise generating a report with the classified protozoa identified and treatment and/or treatment resistance information for each classified or characterized protozoan. Exemplary systems and methods for characterizing, identifying, and/or classifying the protozoa are discussed below.

Exemplary methods of characterizing one or more protozoa includes the step of selecting, by a computer, a digital file comprising one or more digital nucleic acid sequences (e.g., generated using a method described herein), wherein each of the one or more digital nucleic acid sequences corresponds to one or more protozoa to be characterized. The computer segments each of the one or more digital nucleic acid sequences into one or more first portions, performs a set of alignments by comparing the one or more first portions to information stored in a first database, and determines sequence portions from among the one or more first portions that have an alignment match (e.g., within a specified or predetermined range) to the information stored in the first database. Exemplary methods can further include performing, by the computer, a set of alignments by comparing the one or more first portions or one or more second portions to information stored in the first of a second database, determining sequence portions from among the one or more first portions or the one or more second portions that have an alignment match (e.g., within a specified or predetermined range) to the information stored in the second database, and characterizing one or more microorganisms or nucleic acid fragments thereof based on the alignment match to the information stored in one or more of the first database and the second database. Exemplary methods can employ use of one, two, or more databases. In accordance with various aspects of these embodiments, the method can be used to characterize multiple microorganisms (e.g., including protozoa) simultaneously or in parallel, such that multiple microorganisms can be identified in a relatively short amount of time—e.g., preferably in less than forty-eight or less than twenty-four hours or less than 12 hours. Methods can include additional steps of segmenting and using a computer, performing a set of alignments with information stored in a database. These additional steps can include comparing information in the first database, a second database, or other databases. Exemplary methods can also include a step of automatically detecting a sequence run, as disclosed in U.S. patent application Ser. No. 14/196,999, filed Mar. 4, 2014, and entitled METHOD AND KIT FOR CHARACTERIZING MICROORGANISMS.

In accordance with some exemplary embodiments, a digital file comprising one or more digital DNA sequences is (e.g., automatically) selected. The digital file can include a plurality of DNA sequences from the one or more files (e.g., FASTA files) that can comprise a predetermined number of base pairs (bp) or otherwise have a predetermined length. In some implementations, 100 bp may be a preferred number of base pairs at which to set this selection threshold, however, any other number of base pairs that allows for adequate processing and elimination of sequence portions that are unlikely to lead to meaningful analysis may also be selected. For example, greater than or equal to 50 bp, 100 bp, or 150 bp may be used.

The selected DNA sequence file(s) can be segmented into one or more first portions, which may be of about equal size or length. While any number of (e.g., equal or about equal) portions may be used, in some implementations, it may be desirable to match the number of portions to the number of processing cores to be used by a system for processing. For example, when using an analysis computer that has 32 cores, it may be desirable to use 30 of those cores for processing while keeping the remaining two cores in reserve for data management and other processing functions. By way of particular example, it may then be preferable to divide the (e.g., FASTA) sequence file into 30 equal or about equal portions, such that one portion of the file may be processed by each desired processing core.

Once the division of one or more digital DNA sequences into one or more first portions is complete, a set of alignments is performed by comparing the one or more first portions to information stored in a first database. The alignments can be performed using a variety of techniques, including Basic Local Alignment Search Tool (BLAST), OTU, G-BLASTN, mpiBLAST, BLASTX, PAUDA, USEARCH, LAST, BLAT, or other suitable technique. The first database can include a database that includes nucleic acid information (e.g., DNA and/or RNA information) corresponding to one or more types of microorganism—e.g., bacteria, viruses, protozoa, or fungi. By way of examples, the first database can include a protozoa nucleic acid database, such as an 18S rRNA gene database or other protozoa sequence database.

The alignments may in some implementations occur substantially simultaneously. It may also be preferable to perform the alignments using a relatively small comparison window (e.g., 10 bp or 11 bp) as the first database may be relatively small and thus, the processing time does not become prohibitive even with relatively small comparison windows. Exemplary methods can include collating the aggregate results and eliminating any duplicates present. This may be done, for example, when the alignments are complete.

A computer then determines sequence portions from among the one or more first portions that have an alignment match to the information stored in the first database. The step of determining may be based on a predetermined criteria or tolerance for a match.

Each of the one or more digital DNA sequences can optionally be further segmented into one or more second portions. During this optional step, the sequence files can be divided into a second plurality of sequence portions, which may be of equal size and/or the number of portions may be determined by a preferred number of processing cores to be used. In accordance with some exemplary embodiments, the second portions differ or are exclusive of the first portions. The second portions can be compared to information in the first database, to information in a second database, and/or to information in yet another database. Various steps can be repeated in an iterative manner—e.g., wherein a comparison window for determining a match decreases as the number (n) of runs increases. For example, the initial comparison window size can start at 65 bp, and decrease to 40 bp, 25 bp, 10 bp with subsequent runs.

The alignment results can be collated and any duplicates removed. The results can then be checked to determine if all of the sequence file portions were aligned through the running of the alignments. Sequence information can also be compared to another database to, for example, provide further screening, correct a name of a protozoa, determine whether there was a useful alignment, and the like.

A quality of the results of comparisons of matches can be checked by limiting the analysis to sequence portions that have a predetermined length. For example, either a minimum threshold for sequence length could be set such as, for example, a minimum sequence length of 100 bp, or the results may be limited such that only those above which fall into a certain percentage of the longest sequences, for example, the top 100%, 50%, 30%, 20%, 15%, or 10% of all run sequence lengths may be selected on which to base the remaining analysis. By way of one example, the top 90%. 80%. 75%, 70%, 60%, or 50% of sequence lengths can be used. The results can then be tabulated to determine how many matches correspond to each characterized or identified microorganism and any region information can also be tabulated to determine the number of matches for each region analyzed.

A system can then query a database of treatment information that may contain information such as the treatment (e.g., antibiotic, antiviral, antifungal, antiprotozoal) treatment and sensitivity and/or therapy resistance of the treatment(s) corresponding to each identified microorganism and the retrieved information may then be used to generate a report. The output of the report may display information such as, but not limited to: patient information, medical professional information, sample type, collection date, graphical or numerical data relating to one or more characterized or identified microorganisms, a percentage or other numerical indicator of contribution amount of each identified microorganism, a quantitative indicator for a match (e.g., an E-value or % Identity), a description of identified and/or unidentified (novel) microorganisms, and/or treatment sensitivity and/or therapy resistance information.

Exemplary methods of the present disclosure described above may be implemented as one or more software processes executable by one or more processors and/or one or more firmware applications. The processors and/or firmware are configured to operate on one or more general purpose microprocessors or controllers, a field programmable gate array (FPGA), an application specific integrated circuit (ASIC), or other hardware capable of performing the actions describe above. In an exemplary embodiment of the present disclosure, software processes are executed by a CPU in order to perform the actions of the present disclosure. Additionally, the present disclosure is not described with reference to any particular programming language. It will be appreciated that a variety of programming languages may be used to implement the teachings of the disclosure as described herein.

Any of the methods herein may be employed with any form of memory device including all forms of sequential, pseudo-random, and random access storage devices. Storage devices as known within the current art include all forms of random access memory, magnetic and optical tape, magnetic and optical disks, along with various other forms of solid-state mass storage devices. The current disclosure applies to all forms and manners of memory devices including, but not limited to, storage devices utilizing magnetic, optical, and chemical techniques, or any combination thereof.

In some aspects of the present disclosure, the computer-based genomic analysis makes use of a procedural algorithm. By way of particular example, an Ion Sequencing data or other platform data can be imported into CLC Workbench and the sequences sorted. Sequences that are less than 100 bp in length can be removed. The entire data set (e.g., >100 bp) is then BLASTed to a database including know protozoa sequences—e.g., an NT database. The resulting data can be sorted by BLAST hit length. The distribution of the sequence reads from the sequencer can analyzed to determine an appropriate cut-off to obtain a significant number of reads. Less than 20 reads can be deemed not acceptable, for example. Generally, hundreds if not thousands of high quality long reads are included. By way of example, returned species or other taxonomic classification greater than the cut-off can be tabulated for the number of times they occur as a BLAST result. Typically, sequences can be present 5, 10, 25, 50, 100 or more times and can constitute at least 1%, 10%, 15%, 20% or more of the sample to be reported. By way of particular example, about 20% or more contributors with 50 or more sequences can be used. Any sequence that does not meet both of these requirements may not be reported. Depending on the cut-off used, a confidence percentage is applied to the resulting species, genus, or microorganism calls. This data may be presented graphically. In one example, a maximum of six of the top species with a complete listing in tabular format is reported. Treatment (e.g., antibacterial, antifungal, antiviral, and/or antiprotozoal) susceptibilities for each genus/species/microorganism characterized or identified may also be reported. The references for all of the treatment susceptibilities can be listed in the report.

Exemplary reports can include:
1. Patient, physician, and other pertinent test information.
2. The top significant microbial species genus or other taxonomic classification of microbials identified by the sequence analysis.
3. The top significant identified species including Genus specific treatments (e.g., antibiotics, antifungals, antivirals, or antiprotozoals) and any noted treatment resistance for organisms in that Genus. It is important to note that these are not drug sensitivities derived from sequence information, but literature derived suggestions as to what therapies show efficacy in vivo or in vitro. Furthermore, treatments for the Genus may also show up in the noted resistance column, as the results are not mutually exclusive.
4. A Notes section can include performance characteristics of this assay both general and specific to the submitted sample.
5. A listing of the all of the significant identified microbes including total sequence counts and percentages in addition to “Close Match” and “Potential Novel” counts and percentages.
6. Detailed treatment susceptibility with references can be listed for each identified Genus and can be ordered in the order of contribution to the sample. This allows for easy reference to confirm or obtain detailed information about previous literature studying the susceptibility of various bacterial Genera. This section may extend for several pages of detailed reference information.

The steps performed using a computer can be performed using traditional or mobile computerized interfaces or network capable of providing the disclosed processing, querying, and displaying functionalities. Various examples of the disclosed systems and methods may be carried out through the use of one or more computers, processors, servers, databases, and the like. Various examples disclosed herein provide highly efficient computerized systems and methods for characterizing one or more microorganisms or DNA fragments thereof, such as for example, pathogenic protozoa in an efficient and timely manner, such that the systems and methods are suitable for use in clinical settings.

### EXAMPLES

### Example 1

### General DNA Extraction Procedures

Tissues, fluids, other biopsy material, environmental, or industrial material that is suspected of containing bacterial cells are extracted using one of three main methods:

Bone or Tough Tissue Preparation
1. ˜200 mg of bone or tissue is placed in a sterile 50 mL conical tube and 5 mL of molecular grade water is added to the sample.
2. The tissue is sonicated in 5-10 second bursts for a minimum of 5 minutes using a sterile sonicator probe at 10-14 watts.
3. 200 μL of supernatant and any remaining bone/tissue fragments are transferred to a sterile 2 mL screw cap tube and 50-100 μL of 1 mm uneven stainless steel beads, 200 μL of Qiagen Buffer AL, and 20 μL of Proteinase K is added to the sample.
4. The tube is then processed using a percussion based bead homogenizer for 5 minutes at medium speed.
5. 600 μL of the resulting supernatant is run through a inert filter column to remove beads.
6. 200 μL of 100% Ethanol is added to the sample.
7. From here the remaining steps are carried out as described in the Qiagen QIAamp DNA Blood Mini Kit protocol.
8. Final DNA is eluted in 30 μL.
9. Concentration of the extracted DNA is determined by NanoDrop analysis (Thermo Scientific, Wilmington, Del.) of 4 μL.

Soft Tissue Preparation
1. 200 mg of soft tissue and 200 μL of molecular grade water is transferred to a sterile 2 mL screw cap tube and 50-100 μL of 1 mm glass beads, 200 μL of Qiagen Buffer AL, and 20 μL of Proteinase K is added to the sample.
2. The tube is then processed using a percussion based bead homogenizer for 5 minutes at medium speed.
3. ˜600 μL of the resulting supernatant is run through an inert filter column to remove beads.
4. 200 L of 100% Ethanol is added to the sample.
5. From here the remaining steps are carried out as described in the Qiagen QIAamp DNA Blood Mini Kit protocol.
6. Final DNA is eluted in 30 μL.
7. Concentration of the extracted DNA is determined by NanoDrop analysis (Thermo Scientific, Wilmington, Del.) of 4 μL.

Fluid Preparation
1. 200 L of blood or fluid is transferred to a sterile 2 mL screw cap tube and 50-100 μL of 1 mm glass beads, 200 μL of Qiagen Buffer AL, and 20 μL of Proteinase K is added to the sample.
2. The tube is then processed using a percussion based bead homogenizer for 5 minutes at medium speed.
3. ˜400 μL of the resulting supernatant is run through a inert filter column to remove beads.
4. 200 μL of 100% Ethanol is added to the sample.
5. From here the remaining steps are carried out as described in the Qiagen QIAamp DNA Blood Mini Kit protocol.
6. Final DNA is eluted in 30 μL.
7. Concentration of the extracted DNA is determined by NanoDrop analysis (Thermo Scientific, Wilmington, Del.) of 4 μL.

### Example 2

### DNA Purification from Tissues with the QIAamp® DNA Mini Kit

DNA is purified from tissues using the QIAamp® DNA Mini Kit (QIAGEN, Germantown, Md.).

Important points before starting:
All centrifugation steps are carried out at room temperature (15-25° C.).
Use carrier DNA if the sample contains <10,000 genome equivalents.
Avoid repeated freezing and thawing of stored samples, since this leads to reduced DNA size.
Transcriptionally active tissues, such as liver and kidney, contain high levels of RNA which will copurify with genomic DNA. RNA may inhibit some downstream enzymatic reactions, but will not inhibit PCR. If RNA-free genomic DNA is required, include the RNase A digest, as described in step 5a of the protocol.
Things to do before starting:
Equilibrate the sample to room temperature (15-25° C.).
Heat 2 water baths or heating blocks: one to 56° C. for use in step 3, and one to 70° C. for use in step 5.
Equilibrate Buffer AE or distilled water to room temperature for elution in step 11.
Ensure that Buffers AW1 and AW2 have been prepared.
If a precipitate has formed in Buffer ATL or Buffer AL, dissolve by incubating at 56° C.

Procedure

1. Excise the tissue sample or remove it from storage. Determine the amount of tissue. Do not use more than 25 mg (10 mg spleen). Weighing tissue is the most accurate way to determine the amount. If DNA is prepared from spleen tissue, no more than 10 mg should be used. The yield of DNA will depend on both the amount and the type of tissue processed. 1 mg of tissue will yield approximately 0.2-1.2 μg of DNA.

2. Cut up (step 2a), grind (step 2b), or mechanically disrupt (step 2c) the tissue sample. The QIAamp procedure requires no mechanical disruption of the tissue sample, but lysis time will be reduced if the sample is ground in liquid nitrogen (step 2b) or mechanically homogenized (step 2c) in advance.

2a. Cut up to 25 mg of tissue (up to 10 mg spleen) into small pieces. Place in a 1.5 ml microcentrifuge tube, and add 180 μl of Buffer ATL. Proceed with step 3. It is important to cut the tissue into small pieces to decrease lysis time. 2 ml microcentrifuge tubes may be better suited for lysis.

2b. Place up to 25 mg of tissue (10 mg spleen) in liquid nitrogen, and grind thoroughly with a mortar and pestle. Decant tissue powder and liquid nitrogen into 1.5 ml microcentrifuge tube. Allow the liquid nitrogen to evaporate, but do not allow the tissue to thaw, and add 180 μl of Buffer ATL. Proceed with step 3.

2c. Add up to 25 mg of tissue (10 mg spleen) to a 1.5 ml microcentrifuge tube containing no more than 80 μl PBS. Homogenize the sample using the TissueRuptor or equivalent rotor-stator homogenizer. Add 100 μl Buffer ATL, and proceed with step 3. Some tissues require undiluted Buffer ATL for complete lysis. In this case, grinding in liquid nitrogen is recommended. Samples cannot be homogenized directly in Buffer ATL, which contains detergent.

3. Add 20 μl proteinase K, mix by vortexing, and incubate at 56° C. until the tissue is completely lysed. Vortex occasionally during incubation to disperse the sample, or place in a shaking water bath or on a rocking platform. Note: Proteinase K must be used. QIAGEN Protease has reduced activity in the presence of Buffer ATL. Lysis time varies depending on the type of tissue processed. Lysis is usually complete in 1-3 h. Lysis overnight is possible and does not influence the preparation. In order to ensure efficient lysis, a shaking water bath or a rocking platform should be used. If not available, vortexing 2-3 times per hour during incubation is recommended.

4. Briefly centrifuge the 1.5 ml microcentrifuge tube to remove drops from the inside of the lid.

5. If RNA-free genomic DNA is required, follow step 5a. Otherwise, follow step 5b. Transcriptionally active tissues, such as liver and kidney, contain high levels of RNA which will copurify with genomic DNA. RNA may inhibit some downstream enzymatic reactions, but will not inhibit PCR.

5a. First add 4 μl RNase A (100 mg/ml), mix by pulse-vortexing for 15 s, and incubate for 2 min at room temperature. Briefly centrifuge the 1.5 ml microcentrifuge tube to remove drops from inside the lid before adding 200 μl Buffer AL to the sample. Mix again by pulse-vortexing for 15 s, and incubate at 70° C. for 10 min. Briefly centrifuge the 1.5 ml microcentrifuge tube to remove drops from inside the lid. It is essential that the sample and Buffer AL are mixed thoroughly to yield a homogeneous solution. A white precipitate may form on addition of Buffer AL. In most cases it will dissolve during incubation at 70° C. The precipitate does not interfere with the QIAamp procedure or with any subsequent application.

5b. Add 200 μl Buffer AL to the sample, mix by pulse-vortexing for 15 s, and incubate at 70° C. for 10 min. Briefly centrifuge the 1.5 ml microcentrifuge tube to remove drops from inside the lid. It is essential that the sample and Buffer AL are mixed thoroughly to yield a homogeneous solution. A white precipitate may form on addition of Buffer AL, which in most cases will dissolve during incubation at 70° C. The precipitate does not interfere with the QIAamp procedure or with any subsequent application.

6. Add 200 μl ethanol (96-100%) to the sample, and mix by pulse-vortexing for 15 s. After mixing, briefly centrifuge the 1.5 ml microcentrifuge tube to remove drops from inside the lid. It is essential that the sample, Buffer AL, and the ethanol are mixed thoroughly to yield a homogeneous solution. A white precipitate may form on addition of ethanol. It is essential to apply all of the precipitate to the QIAamp Mini spin column. This precipitate does not interfere with the QIAamp procedure or with any subsequent application. Do not use alcohols other than ethanol since this may result in reduced yields.

7. Carefully apply the mixture from step 6 (including the precipitate) to the QIAamp Mini spin column (in a 2 ml collection tube) without wetting the rim. Close the cap, and centrifuge at 6000×g (8000 rpm) for 1 min. Place the QIAamp Mini spin column in a clean 2 ml collection tube, and discard the tube containing the filtrate. Close each spin column to avoid aerosol formation during centrifugation. It is essential to apply all of the precipitate to the QIAamp Mini spin column. Centrifugation is performed at 6000×g (8000 rpm) in order to reduce noise. Centrifugation at full speed will not affect the yield or purity of the DNA. If the solution has not completely passed through the membrane, centrifuge again at a higher speed until all the solution has passed through.

8. Carefully open the QIAamp Mini spin column and add 500 μl Buffer AW1 without wetting the rim. Close the cap, and centrifuge at 6000×g (8000 rpm) for 1 min. Place the QIAamp Mini spin column in a clean 2 ml collection tube, and discard the collection tube containing the filtrate.

9. Carefully open the QIAamp Mini spin column and add 500 μl Buffer AW2 without wetting the rim. Close the cap and centrifuge at full speed (20,000×g; 14,000 rpm) for 3 min.

10. Recommended: Place the QIAamp Mini spin column in a new 2 ml collection tube and discard the old collection tube with the filtrate. Centrifuge at full speed for 1 min. This step helps to eliminate the chance of possible Buffer AW2 carryover.

11. Place the QIAamp Mini spin column in a clean 1.5 ml microcentrifuge tube, and discard the collection tube containing the filtrate. Carefully open the QIAamp Mini spin column and add 200 μl Buffer AE or distilled water. Incubate at room temperature for 1 min, and then centrifuge at 6000×g (8000 rpm) for 1 min.

12. Repeat step 11. A 5 min incubation of the QIAamp Mini spin column loaded with Buffer AE or water, before centrifugation, generally increases DNA yield. A third elution step with a further 200 μl Buffer AE will increase yields by up to 15%. Volumes of more than 200 μl should not be eluted into a 1.5 ml microcentrifuge tube because the spin column will come into contact with the eluate, leading to possible aerosol formation during centrifugation. Elution with volumes of less than 200 μl increases the final DNA concentration in the eluate significantly, but slightly reduces the overall DNA yield. Eluting with 4×100 μl instead of 2×200 μl does not increase elution efficiency. For long-term storage of DNA, eluting in Buffer AE and placing at −20° C. is recommended, since DNA stored in water is subject to acid hydrolysis. Yields of DNA will depend both on the amount and the type of tissue processed. 25 mg of tissue will yield approximately 10-30 μg of DNA in 400 μl of water (25-75 ng/μl), with an A₂₆₀/A₂₈₀ ratio of 1.7-1.9.

### Example 3

### DNA Purification from Blood with the QIAamp® DNA Mini Kit

DNA is purified from blood using the QIAamp® DNA Mini Kit (QIAGEN, Germantown, Md.).

This protocol is for purification of total (genomic, mitochondrial, and viral) DNA from whole blood, plasma, serum, buffy coat, lymphocytes, and body fluids using a microcentrifuge.

Important points before starting:
All centrifugation steps are carried out at room temperature (15-25° C.).
Use carrier DNA if the sample contains <10,000 genome equivalents.
200 μl of whole blood yields 3-12 μg of DNA. Preparation of buffy coat is recommended if a higher yield is required.

Things to do before starting:

Equilibrate samples to room temperature.
Heat a water bath or heating block to 56° C. for use in step 4.
Equilibrate Buffer AE or distilled water to room temperature for elution in step 11.
Ensure that Buffer AW1, Buffer AW2, and QIAGEN Protease have been prepared.
If a precipitate has formed in Buffer AL, dissolve by incubating at 56° C.

Procedure

1. Pipet 20 μl QIAGEN Protease (or proteinase K) into the bottom of a 1.5 ml microcentrifuge tube.

2. Add 200 μl sample to the microcentrifuge tube. Use up to 200 μl whole blood, plasma, serum, buffy coat, or body fluids, or up to 5×106 lymphocytes in 200 μl PBS. If the sample volume is less than 200 μl, add the appropriate volume of PBS. QIAamp Mini spin columns copurify RNA and DNA when both are present in the sample. RNA may inhibit some downstream enzymatic reactions, but not PCR. If RNA-free genomic DNA is required, 4 μl of an RNase A stock solution (100 mg/ml) should be added to the sample before addition of Buffer AL. Note: It is possible to add QIAGEN Protease (or proteinase K) to samples that have already been dispensed into microcentrifuge tubes. In this case, it is important to ensure proper mixing after adding the enzyme.

3. Add 200 μl Buffer AL to the sample. Mix by pulse-vortexing for 15 s. In order to ensure efficient lysis, it is essential that the sample and Buffer AL are mixed thoroughly to yield a homogeneous solution. If the sample volume is larger than 200 μl, increase the amount of QIAGEN Protease (or proteinase K) and Buffer AL proportionally; for example, a 400 μl sample will require 40 μl QIAGEN Protease (or proteinase K) and 400 μl Buffer AL. If sample volumes larger than 400 μl are required, use of QIAamp DNA Blood Midi or Maxi Kits is recommended; these can process up to 2 ml or up to 10 ml of sample, respectively. Note: Do not add QIAGEN Protease or proteinase K directly to Buffer AL.

4. Incubate at 56° C. for 10 min. DNA yield reaches a maximum after lysis for 10 min at 56° C. Longer incubation times have no effect on yield or quality of the purified DNA.

5. Briefly centrifuge the 1.5 ml microcentrifuge tube to remove drops from the inside of the lid.

6. Add 200 μl ethanol (96-100%) to the sample, and mix again by pulse-vortexing for 15 s. After mixing, briefly centrifuge the 1.5 ml microcentrifuge tube to remove drops from the inside of the lid. If the sample volume is greater than 200 μl, increase the amount of ethanol proportionally; for example, a 400 μl sample will require 400 μl of ethanol.

7. Carefully apply the mixture from step 6 to the QIAamp Mini spin column (in a 2 ml collection tube) without wetting the rim. Close the cap, and centrifuge at 6000×g (8000 rpm) for 1 min. Place the QIAamp Mini spin column in a clean 2 ml collection tube, and discard the tube containing the filtrate. Close each spin column in order to avoid aerosol formation during centrifugation. Centrifugation is performed at 6000×g (8000 rpm) in order to reduce noise. Centrifugation at full speed will not affect the yield or purity of the DNA. If the lysate has not completely passed through the column after centrifugation, centrifuge again at higher speed until the QIAamp Mini spin column is empty. Note: When preparing DNA from buffy coat or lymphocytes, centrifugation at full speed is recommended to avoid clogging.

8. Carefully open the QIAamp Mini spin column and add 500 μl Buffer AW1 without wetting the rim. Close the cap and centrifuge at 6000×g (8000 rpm) for 1 min. Place the QIAamp Mini spin column in a clean 2 ml collection tube, and discard the collection tube containing the filtrate. It is not necessary to increase the volume of Buffer AW1 if the original sample volume is larger than 200 μl.

9. Carefully open the QIAamp Mini spin column and add 500 μl Buffer AW2 without wetting the rim. Close the cap and centrifuge at full speed (20,000×g; 14,000 rpm) for 3 min.

10. Recommended: Place the QIAamp Mini spin column in a new 2 ml collection tube and discard the old collection tube with the filtrate. Centrifuge at full speed for 1 min. This step helps to eliminate the chance of possible Buffer AW2 carryover.

11. Place the QIAamp Mini spin column in a clean 1.5 ml microcentrifuge tube, and discard the collection tube containing the filtrate. Carefully open the QIAamp Mini spin column and add 200 μl Buffer AE or distilled water. Incubate at room temperature (15-25° C.) for 1 min, and then centrifuge at 6000×g (8000 rpm) for 1 min. Incubating the QIAamp Mini spin column loaded with Buffer AE or water for 5 min at room temperature before centrifugation generally increases DNA yield. A second elution step with a further 200 μl Buffer AE will increase yields by up to 15%. Volumes of more than 200 μl should not be eluted into a 1.5 ml microcentrifuge tube because the spin column will come into contact with the eluate, leading to possible aerosol formation during centrifugation. Elution with volumes of less than 200 μl increases the final DNA concentration in the eluate significantly, but slightly reduces the overall DNA yield. For samples containing less than 1 μg of DNA, elution in 50 μl Buffer AE or water is recommended. Eluting with 2×100 μl instead of 1×200 μl does not increase elution efficiency. For long-term storage of DNA, eluting in Buffer AE and storing at −20° C. is recommended, since DNA stored in water is subject to acid hydrolysis. A 200 μl sample of whole human blood (approximately 5×106 leukocytes/ml) typically yields 6 μg of DNA in 200 μl water (30 ng/μl) with an A260/A280 ratio of 1.7-1.9.

### Example 4

### Purification of DNA from PCR Reactions

After barcoding and amplification of the extracted DNA, the resulting DNA reactions are purified to remove extraneous DNA sequences that are not the targets for sequencing with standard gel electrophoresis and gel extraction. Gel extraction is performed using the QiaPrep Gel Extraction Mini kit (QIAGEN, Germantown, Md.).

### Example 5

### IonSphere Particle Labeling

All purified DNA samples from the PCR reactions are pooled together in equimolar ratios determined by NanoDrop (Thermo Scientific, Wilmington, Del.) and the known DNA fragment sizes. The pooled library is diluted to precisely 0.08 pM and used as the DNA template for the OneTouch IonSphere Particle Labeling protocol as listed in the Ion OneTouch 200 Template Kit v2 DL (Pub# MAN0007112, Revision: 5.0) in conjunction with the Ion OneTouch 200 Template Kit v2 DL kit.

The OneTouch IonSphere Particle (ISP) Labeling protocol is followed with a few modifications to the “Add Ion OneTouch Reaction Oil” loading step and the “Recover the Template-Positive ISPs” step. The changes are as follows:

Add Ion OneTouch Reaction Oil

Add Ion OneTouch™ Reaction Oil through the sample port:

a. Set a P1000 pipette to 750 μL, and attach a new 1000-μL tip to the pipette.

b. Fill the tip with 750 μL of Reaction Oil.

c. Insert the tip firmly into the sample port so that the tip is perpendicular to the Ion OneTouch™ Plus Reaction Filter Assembly and fully inserted into the sample port to form a tight seal.

d. Gently pipet 750 μL of the Reaction Oil through the sample port. Keep the plunger of the pipette depressed to avoid aspirating solution from the Ion PGM™ OneTouch Plus Reaction Filter Assembly

e. With the plunger still depressed, remove the tip from the sample port, then appropriately discard the tip.

f. Set the P1000 pipette to 750 μL, and attach a new 1000-μL tip to the pipette.

g. Fill the tip with 750 μL of Reaction Oil.

h. Insert the tip firmly into the sample port so that the tip is perpendicular to the Ion OneTouch™ Plus Reaction Filter Assembly and fully inserted into the sample port to form a tight seal.

i. Gently pipet 750 μL of the Reaction Oil through the sample port, then keep the plunger of the pipette depressed.

j. With the plunger still depressed, remove the tip from the sample port, then appropriately discard the tip.

k. If necessary, gently dab a Kimwipes® disposable wiper around the ports to remove any liquid.

Recover the Template-Positive ISPs

1. At the end of the run, ensure that you centrifuged the samples. (Ensure that you have touched Next on the Centrifuge screen to centrifuge the samples and that the home screen displays after the centrifugation.)

2. Immediately after the centrifuge stops, remove and discard the Recovery Router.

3. Carefully remove both Recovery Tubes from the instrument, and put the two Recovery Tubes in a tube rack. You may see some cloudiness in the tube, which is normal.

4. Label a new 1.5-mL LoBind Tube for the template-positive ISPs.

5. Use a pipette to remove all but ˜100 μL of Ion OneTouch™ Recovery Solution from each Ion OneTouch™ Recovery Tube. Do not disturb the pellet of template-positive ISPs.

6. Add 1 mL of Ion OneTouch Wash solution to one Recovery Tube with the ISP pellet and resuspend the pellet by gently pipetting up and down.

7. Transfer the Ion OneTouch Wash solution and resuspended ISPs to the other Recovery Tube and resuspend the pellet by gently pipetting up and down.

8. Transfer the ˜1.2 mL suspension to the new labeled tube.

STOPPING POINT The template-positive ISPs with Ion OneTouch™ Wash Solution may be stored at 2° C. to 8° C. for up to 3 days. After storage, proceed to step 10.

IMPORTANT! Do not store the recovered ISPs in Ion OneTouch™ Recovery Solution.

9. Centrifuge the template-positive ISP suspension for 2.5 minutes at 15,500×g.

10. Remove all but 100 μL of supernatant.

11. Vortex the pellet for 30 seconds to completely resuspend the template-positive ISPs.

12. (Optional) Assess the quality of the unenriched, template-positive ISPs.

13. Enrich the template-positive ISPs.

### Example 6

### IonSphere Particle Enrichment and DNA Sequencing

IonSphere Particle Enrichment

The IonSphere Particle Enrichment protocol is performed as listed in the Ion OneTouch 200 Template Kit v2 DL (Pub# MAN0007112, Revision: 5.0) in conjunction with the Ion OneTouch 200 Template Kit v2 DL kit (Life Technologies, Carlsbad, Calif.).

DNA Sequencing

The DNA Sequencing protocol is performed as listed in the Ion PGM Sequencing Kit manuals for the appropriate sequencing length kit in conjunction with the Ion PGM Sequencing Kits. The only variation to the protocol is a modification of the total flow cycle numbers whereby the total flow cycle number is increased by 80 flows above the kit specifications.

### Example 7

### Computer-Based Genomic Analysis

Once sequencing is complete, individually barcoded sequence sets may be downloaded from the Ion Torrent Browser interface. These are imported as FASTQ files into CLC Workbench. Each sequence set is then processed according to the following steps:

1. Sequences of a specific barcode are length selected and only 100 bp length sequences or greater are retained.

2. These sequences are BLASTed against a local 16S database of known, named, and non-redundant Eubacteria.

3. The resulting BLAST results are size sorted.

4. A size cut-off is selected for each BLAST results based on three factors.

a. Distribution of the reads obtained for that given barcode and the first “cluster” of sequence read lengths is selected with the cut-off as high as possible to include this sequence cluster.

b. If no cluster of sequences is apparent then approximately 100 of the longest sequences are selected for reporting.

c. Sequences less than 100 bp are not used for reporting.

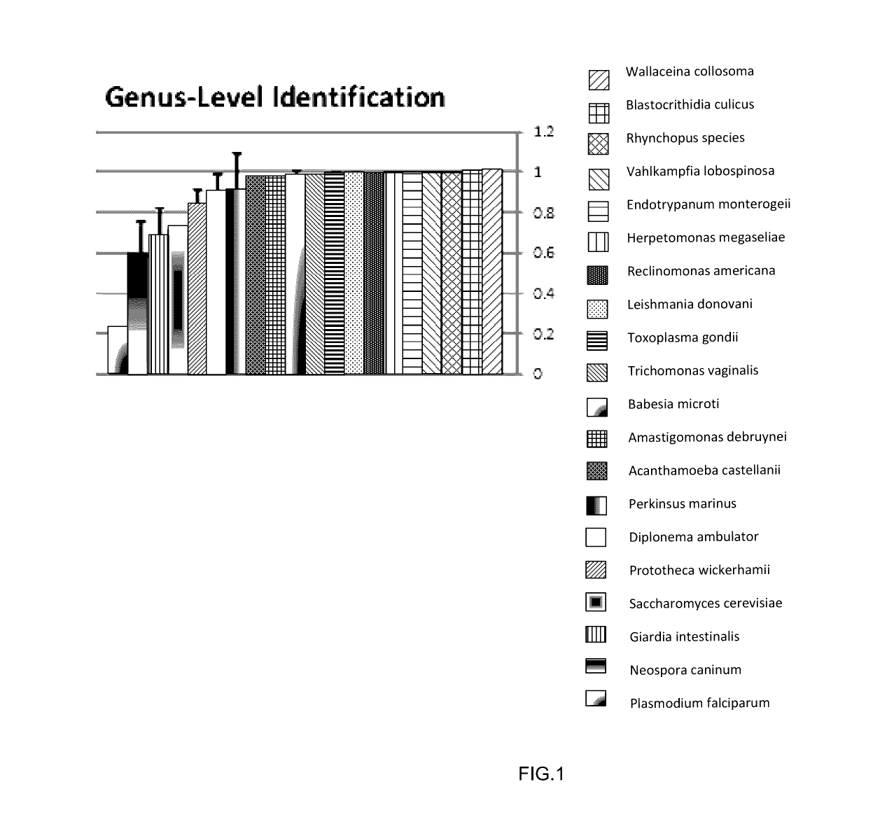

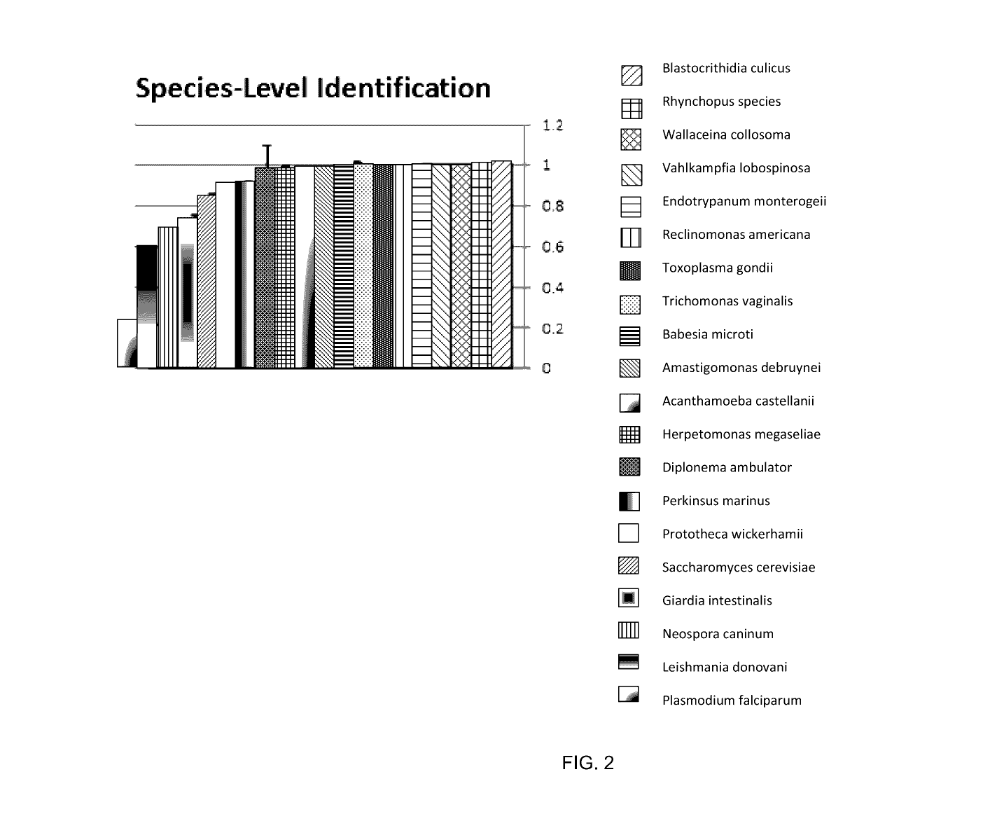

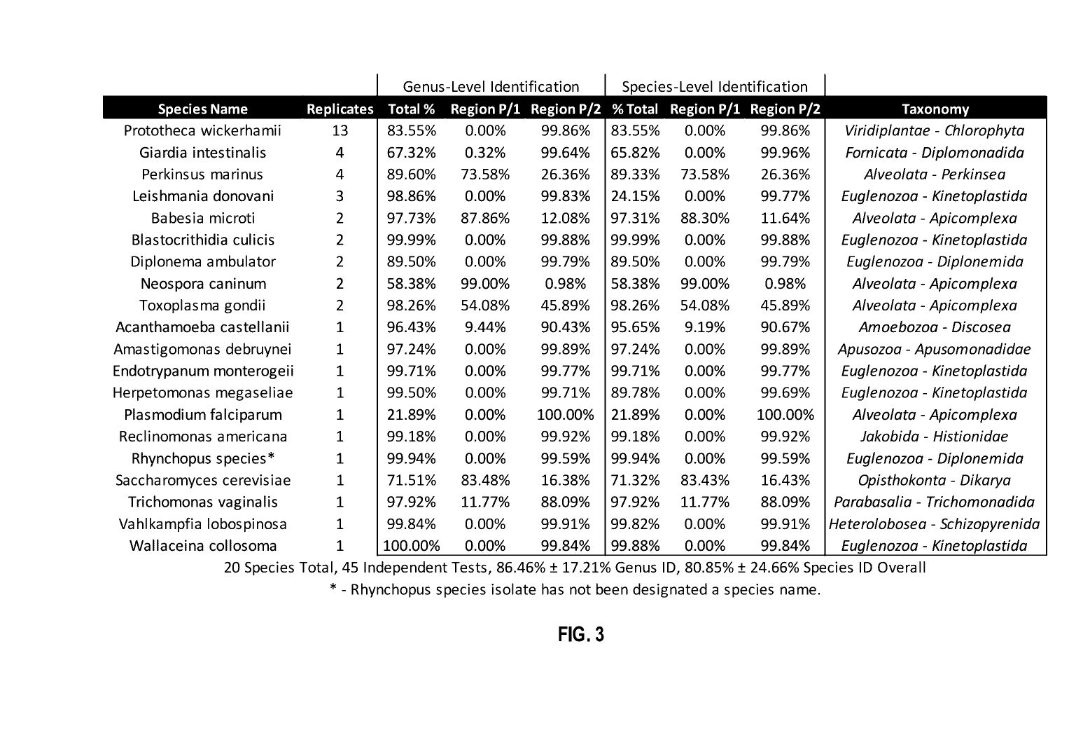

FIGS. 1 and 2 illustrate control runs using methods in accordance with exemplary embodiments of the disclosure, illustrating effectiveness of identification obtained over several replicates and across a large number of species. In the illustrated example, over 20 species in this capacity with over 45 independent replicates for our single species were used for the challenges. Sequencing of region 1 and region 2 of the 18S rRNA gene was done to obtain broad taxonomic coverage. As shown, a large level of sequencing diversity, ranging from plants (Viridiplantae) through all of the major groups of protozoa and into single cell fungi (Opisthokonta) can be characterized using techniques described herein. On average these organisms were correctly detected 86.46% of the time to the genus level. This is on a sequence by sequence basis, so this means that our reports will show 86% of the sequence reads as correctly assigning the genus. Genus identification of this level exceeds currently available technologies in both accuracy and rapidity. The remaining incorrectly assigned species sequences almost exclusively due to extremely closely related members are being identified instead of the intended targeted organism. An example would be how a closely related organism to Leishmania donovanii (L. amazoniensis) is sometimes called by the system, especially when the average read length is on the shorter side. FIGS. 1 and 2 show the relative percentage identification at the genus and species levels in addition to the standard deviation for those instances where we have replicates. Overall going from the species-level from the genus-level we did not lose that much average detection capability (80% from 86%). FIG. 3 provides additional information regarding the control runs.

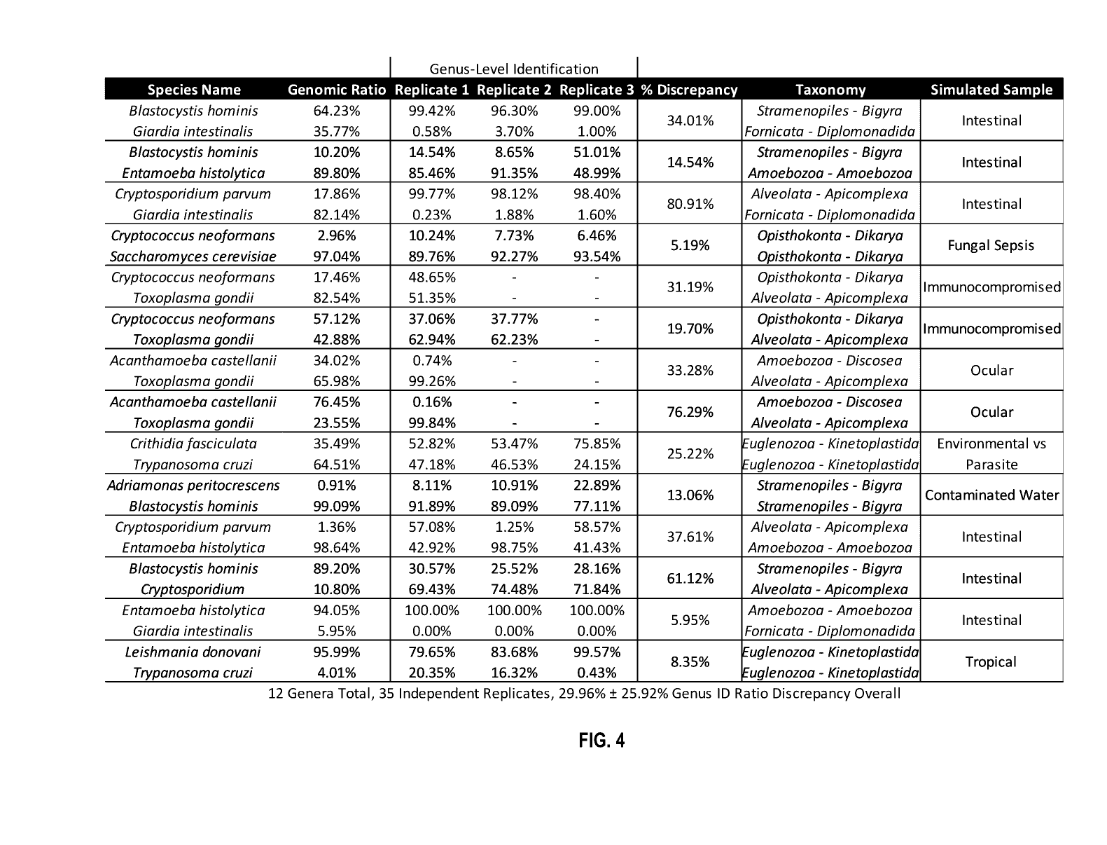

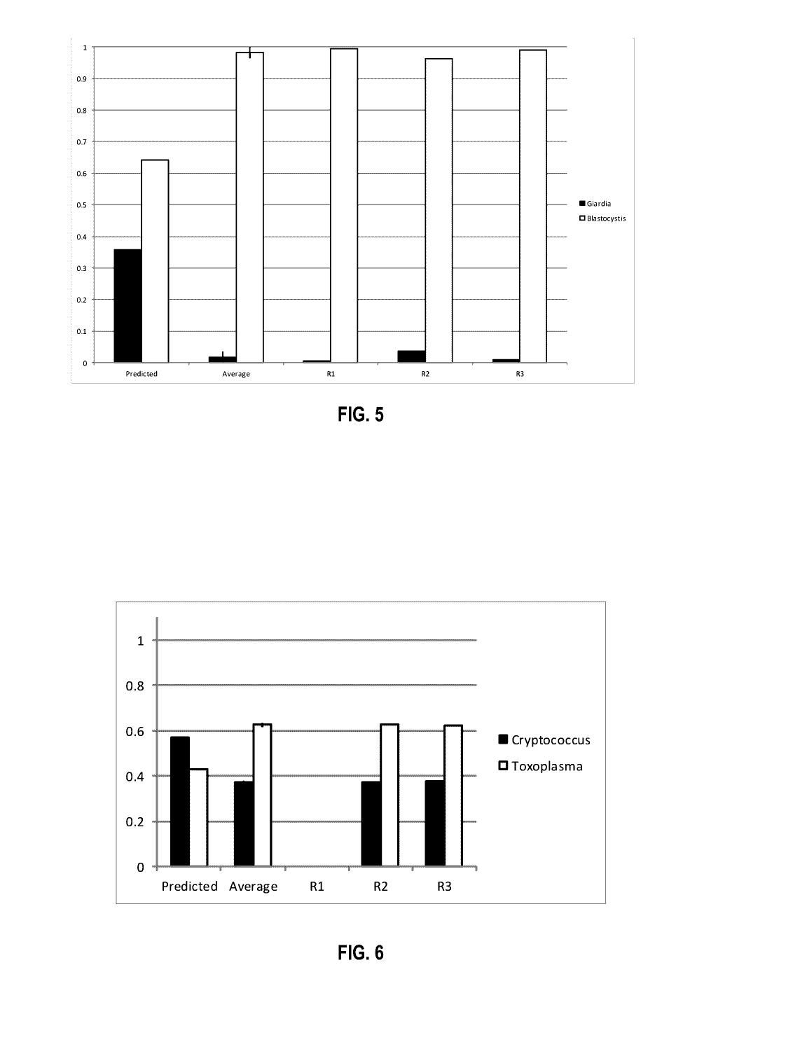

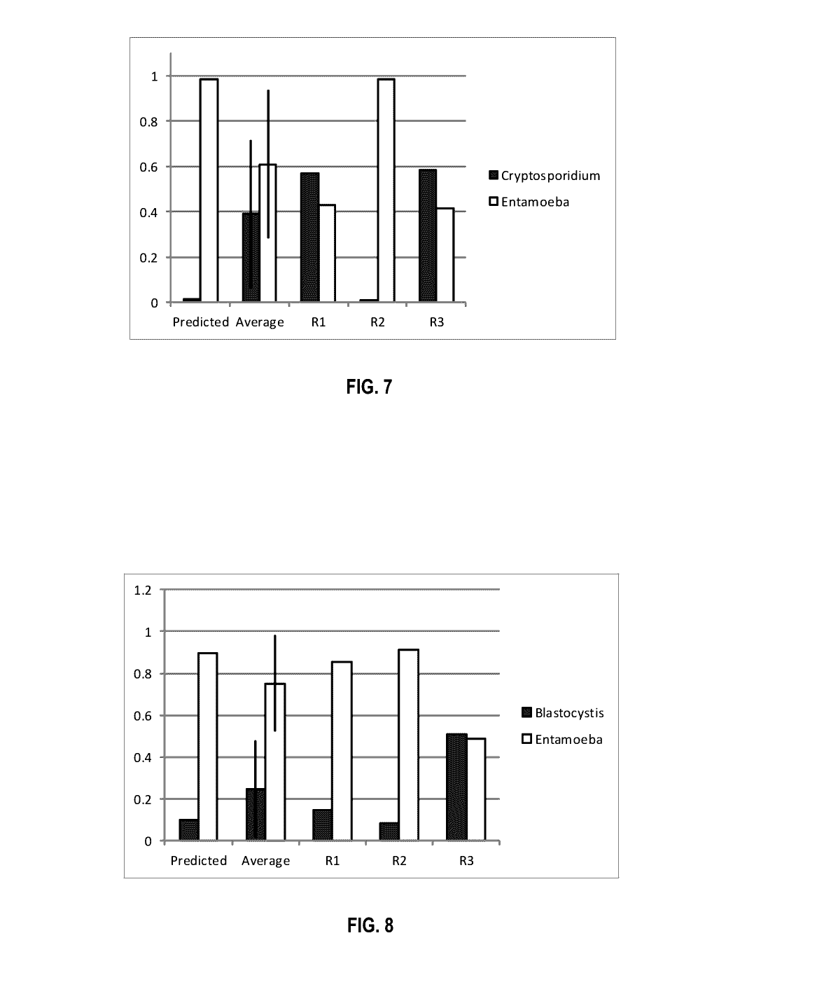

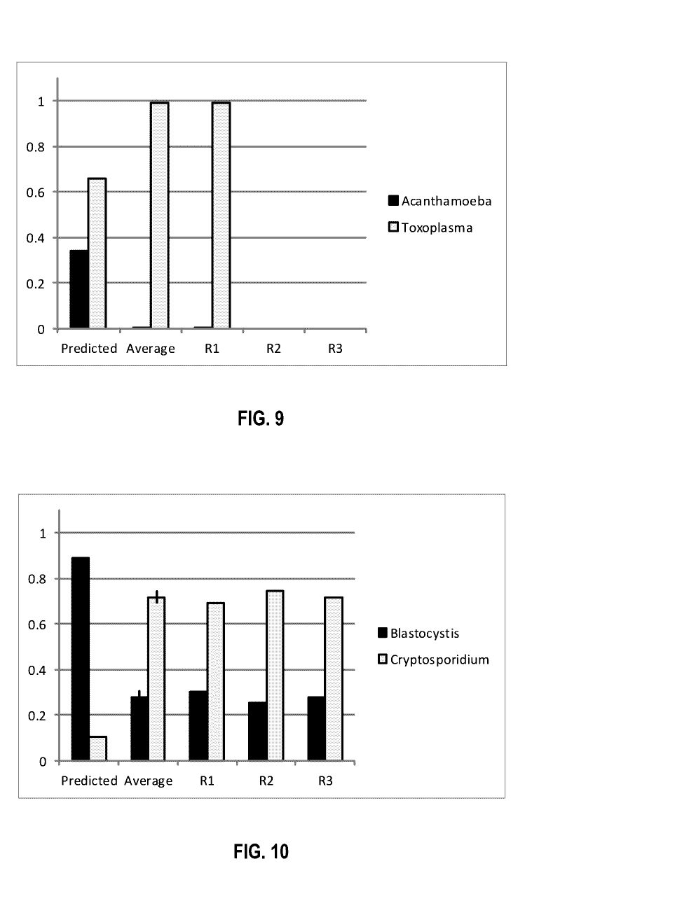

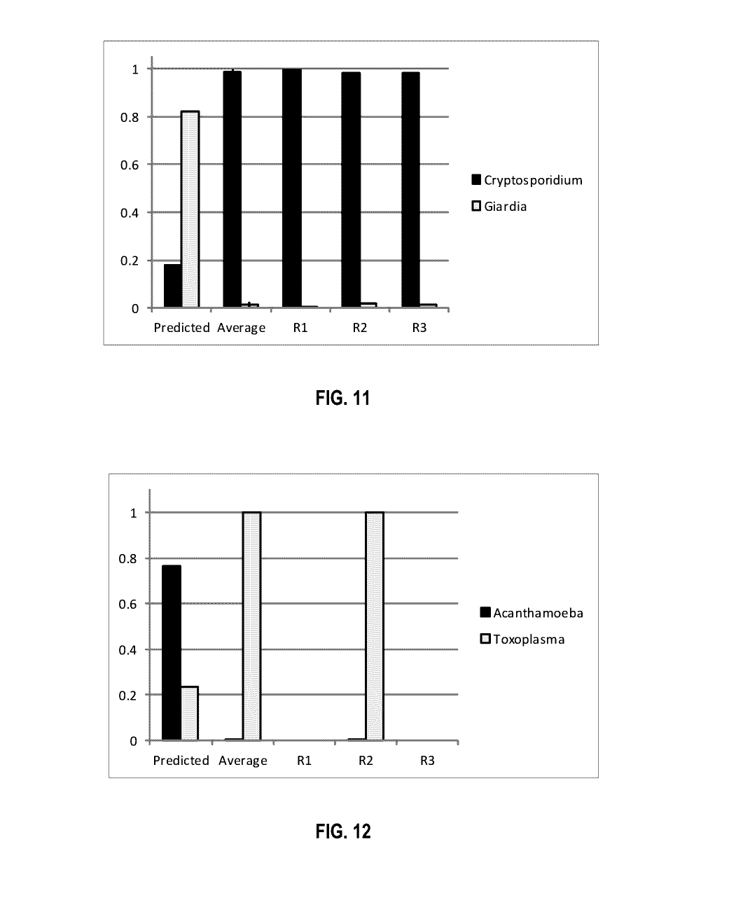

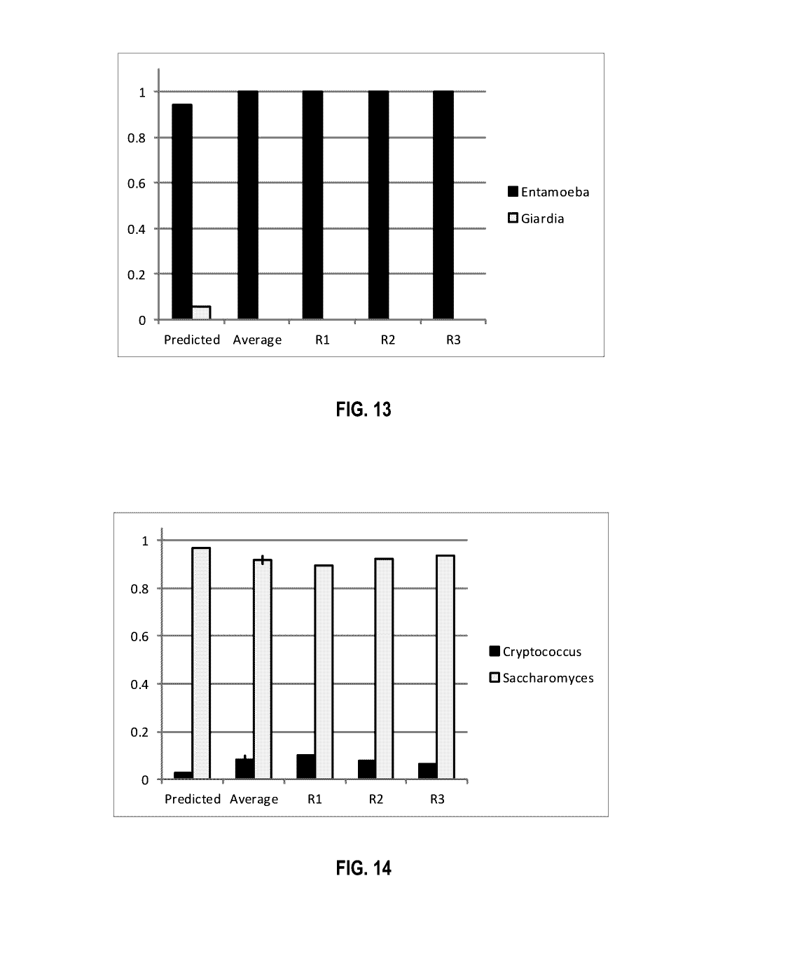

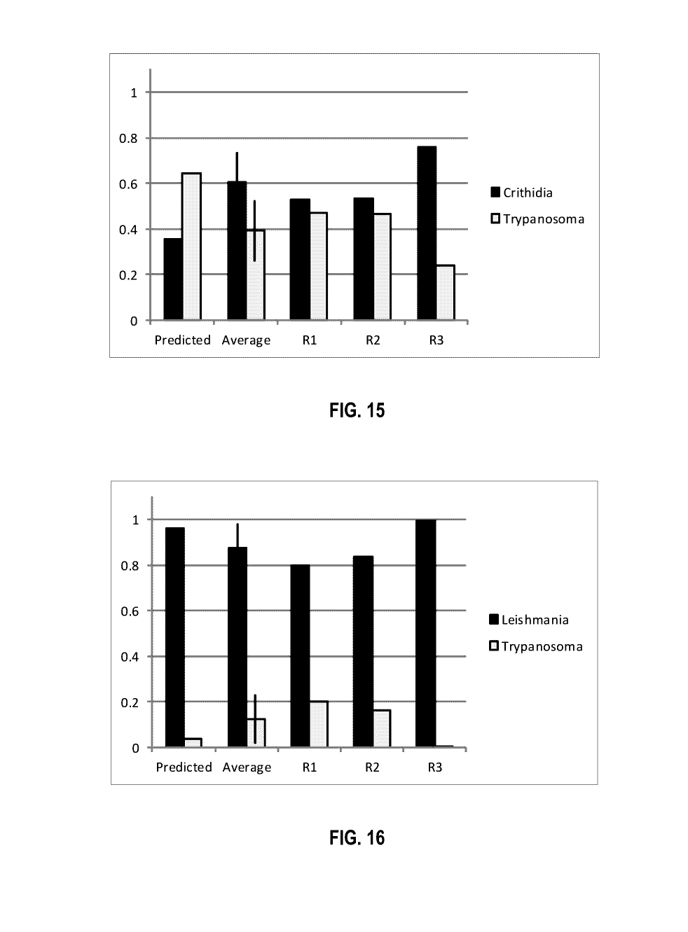

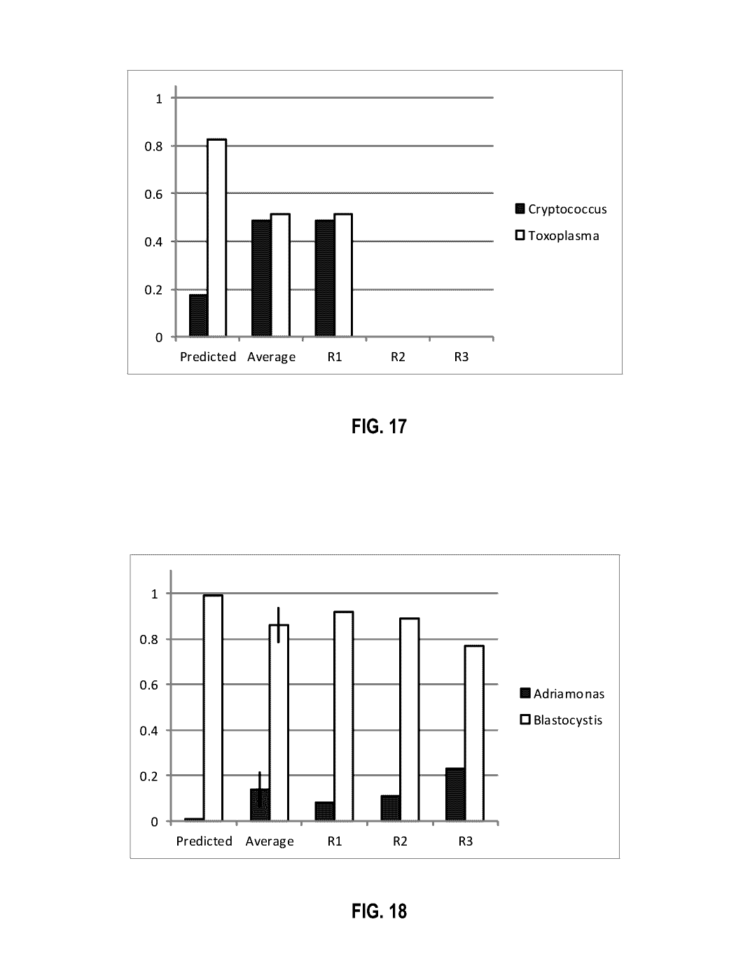

FIGS. 4-18 illustrate results obtained using compositions, methods, and kits as described herein. FIG. 4 illustrates a combination of experiments performed with different organisms and the expected genomic ratios in simulated sample types (i.e., placed where both of these organisms could or might occur simultaneously or the reason for testing them in combination). The recovered genomic ratios are presented for each replicate and the associated % discrepancy observed. The results indicate that we don't see orders of magnitude issues with detecting combinations of organisms that might exist together. These tests included additional organisms that were not tested individually. FIGS. 5-18 illustrate individual graphs summarizing each individual challenge test showing the relative ratios of each organisms that were observed.

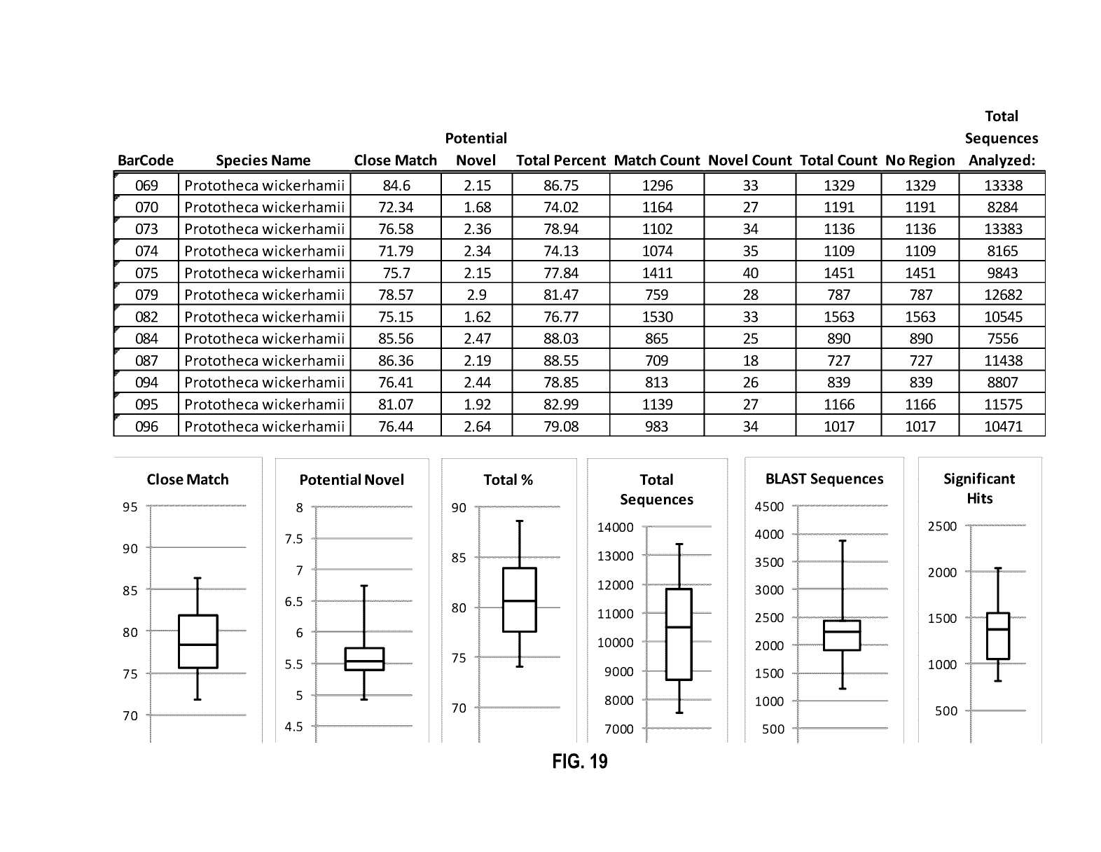

FIG. 19 illustrates the stability of sequencing the same organism over and over in this assay across multiple barcodes. This study illustrates that 1) one can detect Prototheca which is a very unusual infectious plant 2) the barcodes do not significantly influence the outcome of the sequencing, and 3) the stability of the results to arrive at the same answer repeatedly.

The table below illustrates that the methods described herein can detect approximately 19.88 cells per mL of Rhynchopus species in blood samples and 3944.57 cells per mL of Diplonema ambulatory in the samples. This data indicates that the methods can detect organisms as clinically relevant ranges.

Rhynchopus species - Probit Analysis for Limit of Detection

| cells/mL | % Genus ID | Seq Efficiency | log₁₀ | Probit | % Detection |
|---|---|---|---|---|---|
| 6666.67 | 99.94% | 53.25% | 3.8 | 14.0 | 100.00% |
| 6666.67 | 95.44% | 4.97% | | | |
| 6666.67 | 91% | 3.88% | | | |
| 666.67 | 53.70% | 1.17% | 2.8 | 14.0 | 100.00% |
| 666.67 | 50.94% | 5.48% | | | |
| 66.67 | 6.85% | 0.43% | 1.8 | 14.0 | 100.00% |
| 66.67 | 5.78% | 0.11% | | | |
| 6.67 | 0.62% | 0.05% | 0.8 | 0.0 | 0.00% |
| 6.67 | 0.29% | 0.02% | | | |
| 0.67 | 0.00% | 0.00% | −0.2 | 0.0 | 0.00% |
| 0.67 | 0.29% | 0.02% | | | |
| 0.00 | 0.00% | 0.00% | — | 0.0 | 0.00% |

95% Detection at log₁₀ 1.2985 (Probit 6.64) = 19.88 cells/mL

Diplonema ambulator - Probit Analysis for Limit of Detection

| cells/mL | % Genus ID | Seq Efficiency | log₁₀ | Probit | % Detection |
|---|---|---|---|---|---|
| 4166.67 | 47.77% | 24.28% | 3.6 | 6.0 | 83.33% |
| 4166.67 | 0.00% | 0.00% | | | |
| 4166.67 | 93.11% | 24.18% | | | |
| 4166.67 | 96.40% | 26.34% | | | |
| 4166.67 | 95.28% | 8.24% | | | |
| 4166.67 | 96.24% | 39.75% | | | |
| 416.67 | 18.17% | 6.73% | 2.6 | 5.3 | 60.00% |
| 416.67 | 25.11% | 7.90% | | | |
| 416.67 | 52.01% | 5.98% | | | |
| 416.67 | 24.05% | 3.31% | | | |
| 416.67 | 10.44% | 1.83% | | | |
| 41.67 | 0.00% | 0.00% | 1.6 | 4.2 | 20.00% |
| 41.67 | 2.83% | 0.36% | | | |
| 41.67 | 6.98% | 0.39% | | | |
| 41.67 | 0.20% | 0.10% | | | |
| 41.67 | 0.00% | 0.00% | | | |
| 4.17 | 0.16% | 0.05% | 0.6 | 0.0 | 0.00% |
| 4.17 | 0.30% | 0.04% | | | |
| 4.17 | 0.29% | 0.02% | | | |
| 4.17 | 0.00% | 0.00% | | | |
| 4.17 | 0.00% | 0.00% | | | |
| 0.00 | 0.00% | 0.00% | — | 0 | 0.00% |
| 0.00 | 0.00% | 0.00% | | | |
| 0.00 | 0.00% | 0.00% | | | |
| 0.00 | 0.00% | 0.00% | | | |
| 0.00 | 0.00% | 0.00% | | | |
| 0.00 | 0.00% | 0.00% | | | |
| 0.00 | 0.00% | 0.00% | | | |

95% Detection at log₁₀ 3.596 (Probit 6.64) = 3944.57 cells/mL

Additional examples of the disclosure include:

1. A method of characterizing one or more protozoa, the method comprising:

generating a plurality of nucleic acid segments from a sample using one or more degenerate primers to form a pool of nucleic acid segments having a target region;

sequencing the pool of nucleic acid segments to form sequences; and

using a computer, characterizing the one or more protozoa.

2. The method of example 1, wherein the step of characterizing comprises identifying the protozoa or the nearest known protozoa in a library.
3. The method of any of examples 1-2, wherein the step of generating a plurality of nucleic acid segments comprises polymerase chain reaction.
4. The method of any of examples 1-3, wherein the step of generating comprises forming a pool of nucleic acid segments with one or more conserved regions.
5. The method of example any of examples 1-4, wherein the step of generating comprises forming a pool of nucleic acid segments with one or more semi-conserved regions.
6. The method of any of examples 1-5, wherein the step of generating comprises using a forward primer and a reverse primer to amplify a nucleic acid segment corresponding to a section of region of an 18S rRNA gene.
7. The method of any of examples 1-6, wherein the step of generating comprises using a first forward primer and a first reverse primer to amplify a nucleic acid segment corresponding to a section of first region of an 18S rRNA gene and a second forward primer and a second reverse primer to amplify a nucleic acid segment corresponding to a section of a second region of a 18S rRNA gene. The first and second regions can be amplified at the same time.
8. The method of any of examples 1-7, wherein the step of characterizing comprises:

selecting, by the computer, a digital file comprising the sequences;

segmenting, by the computer, each sequences into one or more first portions;

performing, by the computer, a set of alignments by comparing the one or more first portions to information stored in a first database; and

determining, by the computer, sequence portions from among the one or more first portions that have an alignment match within a predetermined limit to the information stored in the first database.

9. The method of example 8, further comprising the step of:

performing, by the computer, a set of alignments by comparing the one or more first portions or one or more second portions to information stored in the first database or a second database; and

characterizing one or more protozoa based on the alignment match to the information stored in one or more of the first database and the second database. The characterization can be corrected if needed.

10. A method of characterizing one or more protozoa, the method comprising:

preparing a nucleic acid library from a sample to form a plurality of nucleic acid segments having one or more of conserved and semi-conserved regions;

sequencing the nucleic acid segments; and

using a computer, characterizing the one or more protozoa.

11. The method of example 10, wherein the nucleic acid library is prepared by polymerase chain reaction.
12. The method of any of examples 1-11, wherein one or more of the plurality of nucleic acid segments comprises one or more targeted conserved or semi-conserved regions.
13. The method of any of examples 1-12, wherein a duration of the step of sequencing is about 12 hours or less.
14. The method of any of examples 1-13, further comprising a step of generating a report indicating one or more likely protozoa present in a sample based on the step of characterizing.
15. The method of any of examples 1-14, wherein two or more microorganisms are characterized.
16. A method of characterizing one or more protozoa comprising:

forming a plurality of nucleic acid segments having one or both of targeted conserved regions and targeted semi-conserved regions;

characterizing the one or more protozoa based on the plurality of nucleic acid segments; and

providing information about one or more of the identity, taxonomy, and relative contribution of the one or more protozoa in a sample.

17. The method of example 16, wherein the step of forming comprises using a forward primer and a reverse primer to amplify segments corresponding to a section of a region of a 18S rRNA gene.
18. The method of any of examples 1-17, wherein the forward primer comprises one or more degenerate bases.
19. The method of example 16, wherein the step of forming comprises using a first forward primer and a first reverse primer to amplify a nucleic acid segment corresponding to section of a first region of a 18S rRNA gene and a second forward primer and a second reverse primer to amplify a segment of nucleic acid corresponding to a section of second region of an 18S rRNA gene.
20. The method of any of examples 1-19, wherein the second forward primer comprises

(SEQ ID NO: 3)
RYGATYAGABACCVYYGTADTC.

21. The method of any of examples 1-20, wherein the step of generating or the step of generating, preparing, or forming comprises use of a primer including an artificial or a non-canonical base.
22. The method of any of examples 1-21, further comprising a step of comparing properties other than a sequence to information in a database.
23. The method of any of examples 1-22, wherein the method comprises metagenomics or community profiling testing.
24. The method of any of examples 1-23, wherein the sample is a clinical sample.
25. The method of any of examples 1-24, wherein the sample is an agricultural sample.
26. A detection method by sequencing capable of characterizing one or more protozoa, the method comprising:

a) generating a plurality of nucleic acid sequence segments by polymerase chain reaction from a sample;

b) sequencing the resulting pool of nucleic acids;

c) identifying the nearest species for the plurality of nucleic acid sequence segments using a computer-based analysis method.

27. A method of identifying a plurality of microorganisms in a biological sample comprising:

a) preparing a DNA library from a sample

b) sequencing conserved or semi-conserved gene sequences by Next Generation DNA sequencing

c) identifying the species or nearest species of the plurality of microorganisms using a computer-based analysis method

28. A method of examples 1-27, wherein the sample is purified nucleic acides from a biological sample containing one or more putative microorganisms.
29. The method of example 28, wherein the DNA library is prepared by polymerase chain reaction.
30. The method of examples 1-29, wherein the DNA library or pool of nucleic acids contain multiple targeted conserved or semi-conserved regions.
31. The method of examples 1-30, wherein the Next Generation DNA sequencing is performed under 12 hours.
32. The method of examples 1-31, wherein the protozoa are identified by a computer-based analysis method that automatically processes the sequence information.
33. The method of example 32, wherein the identified protozoa are presented in a clinical report.
34. A method of any of examples 1-33, where in the plurality of nucleic acid sequence segments or plurality of microorganisms represent organisms across high level or phylum taxonomic groups.
35. The method of any of examples 1-34, where in the taxonomic groups consist of Alveolata, Amoebozoa, Ancyromonadida, Apusomonadida, Apusozoa, Ascomycota, Basidiomycota, Bigyra, Bikonts, Breviata, Centroheliozoa, Cercozoa, Choanoflagellida, Chromalveolata, Chromerida, Chromista, Ciliophora, Collodictyonidae, Corallochytrium, Cryptophyta, Dimorpha, Dinoflagellata, Discoba, Eccrinales, Euglenozoa, Excavata, Excavate, Fonticula, Foraminifera, Fornicata, Fungi, Glaucocystophyceae, Hacrobia, Hapophyta, Haptophyceae, Hemimastigida, Heterokontophyta, Heterolobosea, Ichthyosporea, Jakobida, Katablepharidophyta, Loukozoa, Malawimonadidae, Mantamonas, Metamonada, Metromonas, Microheliella, Ministeria, Myxozoa, Nucleariidae, Opisthokonta, Oxymonadida, Palpitomonas, Palustrimonas, Parabasalia, Percolozoa, Perkinsozoa, Picozoa, Preaxostyla, Radiolaria, Retaria, Rhizaria, Rhodophyta, Stramenopiles, Subulatomonas, Telonemia, Telonemida, Trebouxiophyceae, Trimastix, Tsukubamonadidae, and Unikonts.
36. The method of examples 1-35, wherein the plurality of nucleic acid sequence segments plurality of microorganisms include members from one or more of the group consisting of Giardia species, Toxoplasma species, Babesia species, Leishmania species, Trypanosoma species, Entamoeba species, Cryptosporidium species, Perkinsus species, Acanthamoeba species, Trichomonas species, Blastocystis species, Cyclospora species, Theileria species, Pneumocystis species, Naegleria species, Euglena species, Endotrypanum species, Reclinomonas species, Balamuthia species, Prototheca species, Saccharomyces species, Kluveromyces species, Cyclophora species, Eimeria species, Goussia species, Diplonema species, Enteromonas species, Blastomyces species, Coccidioides species, Histoplasma species, Paracoccidioides species, Sporothrix species, Neospora species, Thecamonas species, Crithidia species, Blastocrithidia species, Leptomonas species, Herpetomonas species, Colpoda species, and Rhynchopus species.
37. A method if claims 1-36, where in groups of these microorganisms or groups of identified sequence segments may be identified simultaneously.
38. A method whereby characterizing multiple specifically selected conserved or semi-conserved DNA sequences yields useful information about the identity, taxonomy, and relative contribution of one or more organisms in a sample.
39. A method of example 38, wherein characterizing occurs by Next Generation DNA sequencing and computer analysis.
40. A method of examples 38-39, wherein conserved or semi-conserved DNA sequences are generated by Polymerase Chain Reaction (PCR).
41. A method of examples 38-40, wherein characterizing includes generating a clinical report.
42. A method examples 38-41, wherein organisms include microorganisms.
43. A method of examples 38-42, wherein the organisms include protozoa.
44. A method of examples 38-43, wherein characterizing occurs within 12 hours.
45. A method examples 38-44, wherein the sample is a biological sample.
46. A method examples 38-45, wherein the sample is a clinical sample.
47. A method examples 38-46, wherein primers are used to generate the conserved or semi-conserved DNA sequences.
48. A kit comprising a first primer to prime a first region of first region of an 18S rRNA gene and a second primer to prime a second region of an 18S rRNA gene.
49. The kit of example 48, wherein the first primer includes one or more degenerate bases.
50. The kit of any of examples 48-49, wherein the first primer includes one or more artificial and/or non-canonical bases.
51. The kit of any of examples 48-49, wherein the second primer includes

RYGATYAGABACCVYYGTADTC.

The aspects/implementations outlined here, and many others, will become readily apparent to those of ordinary skill in the art from this disclosure. Those of ordinary skill in the art will readily understand the versatility with which this disclosure may be applied.

In places where the description above refers to particular implementations of compositions and methods for detecting protozoa, it should be readily apparent that a number of modifications may be made without departing from the spirit thereof and that these implementations may be alternatively applied. The accompanying CLAIMS are intended to cover such modifications as would fall within the true spirit and scope of the disclosure set forth in this document. The presently disclosed implementations are, therefore, to be considered in all respects as illustrative and not restrictive, the scope of the disclosure being indicated by the appended CLAIMS rather than the foregoing DESCRIPTION. All changes that come within the meaning of and range of equivalency of the CLAIMS are intended to be embraced therein.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes to the extent the contents of such do not conflict with the present disclosure.

The publications discussed herein are provided solely for their disclosure prior to the filing date of the present application. Nothing herein is to be construed as an admission that the present disclosure is not entitled to antedate such publication by virtue of prior invention.

The disclosure is not limited to the particular methodology, protocols and materials described as these can vary. It is also understood that the terminology used herein is for the purposes of describing particular embodiments only and is not intended to limit the scope of the present invention, which is set forth in the appended claims and legal equivalents thereof. Additionally, unless otherwise noted, method steps according to an aspect of the invention may be performed in any sequence possible to achieve the desired result.

What is claimed is:

1. A method of characterizing one or more protozoa, the method comprising:
generating a plurality of nucleic acid segments from a sample using one or more degenerate primers to form a pool of nucleic acid segments having a target region;
sequencing the pool of nucleic acid segments to form sequences; and
using a computer, characterizing the one or more protozoa.

2. The method of claim 1, wherein the step of characterizing comprises identifying the protozoa or the nearest known protozoa in a library.

3. The method of claim 1, wherein the step of generating a plurality of nucleic acid segments comprises polymerase chain reaction.

4. The method of claim 1, wherein the step of generating comprises forming a pool of nucleic acid segments with one or more conserved regions.

5. The method of claim 1, wherein the step of generating comprises forming a pool of nucleic acid segments with one or more semi-conserved regions.

6. The method of claim 1, wherein the step of generating comprises using a forward primer and a reverse primer to amplify a nucleic acid segment corresponding to a section of region of an 18S rRNA gene.

7. The method of claim 1, wherein the step of generating comprises using a first forward primer and a first reverse primer to amplify a nucleic acid segment corresponding to a section of first region of an 18S rRNA gene and a second forward primer and a second reverse primer to amplify a nucleic acid segment corresponding to a section of a second region of a 18S rRNA gene.

8. The method of claim 1, wherein the step of characterizing comprises:
selecting, by the computer, a digital file comprising the sequences;
segmenting, by the computer, each sequences into one or more first portions;
performing, by the computer, a set of alignments by comparing the one or more first portions to information stored in a first database; and
determining, by the computer, sequence portions from among the one or more first portions that have an alignment match within a predetermined limit to the information stored in the first database.

9. The method of claim 8, further comprising the step of:
performing, by the computer, a set of alignments by comparing the one or more first portions or one or more second portions to information stored in the first database or a second database; and
characterizing one or more protozoa based on the alignment match to the information stored in one or more of the first database and the second database.

10. A method of characterizing one or more protozoa, the method comprising:
preparing a nucleic acid library from a sample to form a plurality of nucleic acid segments having one or more of conserved and semi-conserved regions;
sequencing the nucleic acid segments; and
using a computer, characterizing the one or more protozoa.

11. The method of claim 10, wherein the nucleic acid library is prepared by polymerase chain reaction.

12. The method of claim 10, wherein one or more of the plurality of nucleic acid segments comprises one or more targeted conserved or semi-conserved regions.

13. The method of claim 10, wherein a duration of the step of sequencing is about 12 hours or less.

14. The method of claim 10, further comprising a step of generating a report indicating one or more likely protozoa present in a sample based on the step of characterizing.

15. The method of claim 10, wherein two or more microorganisms are characterized.

16. A method of characterizing one or more protozoa comprising:
forming a plurality of nucleic acid segments having one or both of targeted conserved regions and targeted semi-conserved regions;
characterizing the one or more protozoa based on the plurality of nucleic acid segments; and
providing information about one or more of the identity, taxonomy, and relative contribution of the one or more protozoa in a sample.

17. The method of claim 16, wherein the step of forming comprises using a forward primer and a reverse primer to amplify segments corresponding to a section of a region of a 18S rRNA gene.

18. The method of claim 17, wherein the forward primer comprises one or more degenerate bases.

19. The method of claim 16, wherein the step of forming comprises using a first forward primer and a first reverse primer to amplify a nucleic acid segment corresponding to section of a first region of a 18S rRNA gene and a second forward primer and a second reverse primer to amplify a segment of nucleic acid corresponding to a section of second region of an 18S rRNA gene.

20. The method of claim 19, wherein the second forward primer comprises
(SEQ ID NO: 3)
RYGATYAGABACCVYYGTADTC.


SEQUENCE LISTING (41 sequences)

SEQ ID NO: 1 - DNA, 22 bp, Artificial sequence
  Synthetic forward primer
ccatgcatgtctaagtataagc

SEQ ID NO: 2 - DNA, 22 bp, Artificial sequence
  Synthetic reverse primer
cagaaacttgaatgatctatcg

SEQ ID NO: 3 - DNA, 22 bp, Artificial sequence
  Synthetic forward primer
rygatyagabaccvyygtadtc

SEQ ID NO: 4 - DNA, 17 bp, Artificial sequence
  Synthetic reverse primer
cgygttgagtcrrattr

SEQ ID NO: 5 - DNA, 65 bp, Artificial sequence
  Synthetic forward primer
ccatctcatccctgcgtgtctccgactcagttcaattggcgatccatgcatgtctaagtataagc

SEQ ID NO: 6 - DNA, 65 bp, Artificial sequence
  Synthetic forward primer
ccatctcatccctgcgtgtctccgactcagcctactggtcgatccatgcatgtctaagtataagc

SEQ ID NO: 7 - DNA, 65 bp, Artificial sequence
  Synthetic forward primer
ccatctcatccctgcgtgtctccgactcagtctgcctgtcgatccatgcatgtctaagtataagc

SEQ ID NO: 8 - DNA, 65 bp, Artificial sequence
  Synthetic forward primer
ccatctcatccctgcgtgtctccgactcagcgatcggttcgatccatgcatgtctaagtataagc

SEQ ID NO: 9 - DNA, 65 bp, Artificial sequence
  Synthetic forward primer
ccatctcatccctgcgtgtctccgactcagtcaggaatacgatccatgcatgtctaagtataagc

SEQ ID NO: 10 - DNA, 65 bp, Artificial sequence
  Synthetic forward primer
ccatctcatccctgcgtgtctccgactcagcctggttgtcgatccatgcatgtctaagtataagc

SEQ ID NO: 11 - DNA, 65 bp, Artificial sequence
  Synthetic forward primer
ccatctcatccctgcgtgtctccgactcagttggcatctcgatccatgcatgtctaagtataagc

SEQ ID NO: 12 - DNA, 65 bp, Artificial sequence
  Synthetic forward primer
ccatctcatccctgcgtgtctccgactcagcttccataacgatccatgcatgtctaagtataagc

SEQ ID NO: 13 - DNA, 65 bp, Artificial sequence
  Synthetic forward primer
ccatctcatccctgcgtgtctccgactcagttggctggacgatccatgcatgtctaagtataagc

SEQ ID NO: 14 - DNA, 65 bp, Artificial sequence
  Synthetic forward primer
ccatctcatccctgcgtgtctccgactcagtccgacaagcgatccatgcatgtctaagtataagc

SEQ ID NO: 15 - DNA, 65 bp, Artificial sequence
  Synthetic forward primer
ccatctcatccctgcgtgtctccgactcagcggacagatcgatccatgcatgtctaagtataagc

SEQ ID NO: 16 - DNA, 65 bp, Artificial sequence
  Synthetic forward primer
ccatctcatccctgcgtgtctccgactcagttaagcggtcgatccatgcatgtctaagtataagc

SEQ ID NO: 17 - DNA, 45 bp, Artificial sequence
  Synthetic reverse primer
cctctctatgggcagtcggtgatcagaaacttgaatgatctatcg

SEQ ID NO: 18 - DNA, 65 bp, Artificial sequence
  Synthetic forward primer
ccatctcatccctgcgtgtctccgactcagttcaattggcgatrygatyagabaccvyygtadtc

SEQ ID NO: 19 - DNA, 65 bp, Artificial sequence
  Synthetic forward primer
ccatctcatccctgcgtgtctccgactcagcctactggtcgatrygatyagabaccvyygtadtc

SEQ ID NO: 20 - DNA, 65 bp, Artificial sequence
  Synthetic forward primer
ccatctcatccctgcgtgtctccgactcagtctgcctgtcgatrygatyagabaccvyygtadtc

SEQ ID NO: 21 - DNA, 65 bp, Artificial sequence
  Synthetic forward primer
ccatctcatccctgcgtgtctccgactcagcgatcggttcgatrygatyagabaccvyygtadtc

SEQ ID NO: 22 - DNA, 65 bp, Artificial sequence
  Synthetic forward primer
ccatctcatccctgcgtgtctccgactcagtcaggaatacgatrygatyagabaccvyygtadtc

SEQ ID NO: 23 - DNA, 65 bp, Artificial sequence
  Synthetic forward primer
ccatctcatccctgcgtgtctccgactcagcctggttgtcgatrygatyagabaccvyygtadtc

SEQ ID NO: 24 - DNA, 65 bp, Artificial sequence
  Synthetic forward primer
ccatctcatccctgcgtgtctccgactcagttggcatctcgatrygatyagabaccvyygtadtc

SEQ ID NO: 25 - DNA, 65 bp, Artificial sequence
  Synthetic forward primer
ccatctcatccctgcgtgtctccgactcagcttccataacgatrygatyagabaccvyygtadtc

SEQ ID NO: 26 - DNA, 65 bp, Artificial sequence
  Synthetic forward primer
ccatctcatccctgcgtgtctccgactcagttggctggacgatrygatyagabaccvyygtadtc

SEQ ID NO: 27 - DNA, 65 bp, Artificial sequence
  Synthetic forward primer
ccatctcatccctgcgtgtctccgactcagtccgacaagcgatrygatyagabaccvyygtadtc

SEQ ID NO: 28 - DNA, 65 bp, Artificial sequence
  Synthetic forward primer
ccatctcatccctgcgtgtctccgactcagcggacagatcgatrygatyagabaccvyygtadtc

SEQ ID NO: 29 - DNA, 65 bp, Artificial sequence
  Synthetic forward primer
ccatctcatccctgcgtgtctccgactcagttaagcggtcgatrygatyagabaccvyygtadtc

SEQ ID NO: 30 - DNA, 40 bp, Artificial sequence
  Synthetic reverse primer
cctctctatgggcagtcggtgatcgygttgagtcrrattr

SEQ ID NO: 31 - DNA, 32 bp, Artificial sequence
  Synthetic probe
ggataaccgtagtaattctggagctaatacat

SEQ ID NO: 32 - DNA, 37 bp, Artificial sequence
  Synthetic probe
taaactrtaactgwtwtaatgagcywtycgcagttty

SEQ ID NO: 33 - DNA, 26 bp, Artificial sequence
  Synthetic probe
ggagctaatacatgatacaggacccg

SEQ ID NO: 34 - DNA, 38 bp, Artificial sequence
  Synthetic probe
gaatggctcattawawcagttayagtttatttgatgat

SEQ ID NO: 35 - DNA, 33 bp, Artificial sequence
  Synthetic probe
ctacgtggataactgtagtaattctagagctaa

SEQ ID NO: 36 - DNA, 34 bp, Artificial sequence
  Synthetic probe
ttatttgatggtttyytacttggataacccgagt

SEQ ID NO: 37 - DNA, 25 bp, Artificial sequence
  Synthetic probe
ctctggctaatatacgctgaagacc

SEQ ID NO: 38 - DNA, 34 bp, Artificial sequence
  Synthetic probe
tggataaccgyrgtaatwctrkrgctaakacatg

SEQ ID NO: 39 - DNA, 33 bp, Artificial sequence
  Synthetic probe
gtgaaactgcgaatggctcattatatcagttat

SEQ ID NO: 40 - DNA, 32 bp, Artificial sequence
  Synthetic probe
waydgygaaactgcgaatggctcattawawca

SEQ ID NO: 41 - DNA, 26 bp, Artificial sequence
  Synthetic probe
acatcctttccgtgaggtcaggagtt